(19) United States
(12) Patent Application Publication
(10) Pub. No.: US 2021/0361683 A1
(43) Pub. Date: Nov. 25, 2021

(54) COMPOSITIONS AND METHODS FOR THE TREATMENT OF MUSCLE CONTRACTURES

(71) Applicant: Children's Hospital Medical Center, Cincinnati, OH (US)

(72) Inventor: Roger Cornwall, Cincinnati, OH (US)

(21) Appl. No.: 17/052,261

(22) PCT Filed: May 23, 2019
(86) PCT No.: PCT/US2019/033677

§ 371 Date: Nov. 2, 2020

Related U.S. Application Data
(60) Provisional application No. 62/675,814, filed on May 24, 2018.

(51) Int. Cl. A61K 31/69; A61K 38/06; A61K 38/17; A61P 21/06

(52) U.S. Cl. CPC A61K 31/69; A61P 21/06; A61K 38/1709; A61K 38/06

(57) ABSTRACT

Disclosed herein are methods and compositions for the treatment of muscle contractures. In particular, the disclosed methods and compositions may be used to improve longitudinal muscle growth in individuals having muscle contractures, for example, muscle contractures resulting from cerebral palsy or brachial plexus injury. The methods and compositions may employ, for example, the administration of a therapeutic dose of a proteasome inhibitor.

### CROSS-REFERENCE TO RELATED APPLICATIONS

This application claims priority to and benefit of U.S. Provisional Application No. 62/675,814, filed May 24, 2018, the contents of each are incorporated in their entirety for all purposes.

### BACKGROUND

Muscle contractures are a prominent and disabling feature of many neuromuscular disorders, including the two most common forms of childhood neurologic dysfunction: neonatal brachial plexus injury (NBPI) and cerebral palsy (CP). There are currently no treatment strategies to correct the contracture pathology, as the pathogenesis of these contractures is unknown.

### BRIEF SUMMARY

Disclosed herein are methods and compositions for the treatment of muscle contractures. In particular, the disclosed methods and compositions may be used to improve longitudinal muscle growth in individuals having muscle contractures, for example, muscle contractures resulting from cerebral palsy or brachial plexus injury. The methods and compositions may employ, for example, the administration of a therapeutic dose of a proteasome inhibitor

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts muscle stem cell dysregulation during development of neonatal contractures. (A), Immunohistochemistry for Pax7 in biceps from contralateral and 2 weeks after neonatal brachial plexus injury (NBPI). Arrows indicate Pax7+ cells. (B) Quantification of biceps sections immunostained with Pax7 and MyoD antibodies to assess stage of muscle stem cell (MuSC) quiescence and activation. The number of Pax7+ MyoD− (quiescent), Pax7+ MyoD+ (activated), Pax7− MyoD+ (differentiated) cells were normalized to total nuclei. (n=4 for contralateral and NPBI). (C) Experimental scheme for BrdU treatment during the initial 2 weeks after NBPI. (D) Representative images (left) of immunostaining with Pax7 and BrdU antibodies in contralateral and NBPI muscle. Arrows show Pax7+ BrdU+ cells and arrowheads show Pax7+ BrdU− cells. Quantification (right) of proliferating MuSCs (Pax7+ BrdU+) as a percentage of total Pax7+ cells (n=7 for contralateral and NBPI). (E) Representative images (left) showing BrdU+ myonuclei, defined as being BrdU+ and entirely within a dystrophin+ myofiber, as an indicator of myonuclear accretion. White arrows indicate a BrdU+ myonucleus, whereas yellow arrows show a BrdU− myonucleus. Quantification (right) of the percentage of myofibers containing a BrdU+ nucleus (n=7 for contralateral and NBPI). Data are presented as mean±SD. Because all comparisons were done to the contralateral, unoperated forelimbs, statistical analyses were performed with paired, two-tailed Student's t-tests except for (B) where Wilcoxon Signed Rank test was used for Pax7+MyoD+ biceps due to non-normal distributions. *P<0.05, **P<0.01, ***P<0.001. Scale bars, 100 μm.

FIGS. 2A-2I depict reduced myonuclear numbers do not control muscle length or contracture pathology. (2A) Myomaker (Mymk) was deleted in MuSCs to prevent myonuclear accretion. Expression of Mymk in muscle from Mymk^{loxP/loxP} (control) and Mymk^{loxP/loxP}; Pax7CreER (Mymk^{scKO}) at postnatal day (P) 5 after treatment with tamoxifen (Tam.) at P0 (n=4 for control and Mymk^{scKO}). (2B) Representative single myofibers from the extensor digitorum longus (EDL), stained with DAPI, of control and Mymk^{scKO} at P28, following tamoxifen at P0. (2C) Quantification of nuclei per myofiber from the samples in (b) (n=3 for control and Mymk^{scKO}). (2D) DIC images (left) from control and Mymk^{scKO} EDL showing similar sarcomere lengths. Nuclei are outlined in red. Quantification (right) of the myonuclear domain in length, expressed as the number of sarcomeres per nucleus in a 1000 μm segment of the myofiber. (n=3 for control and) Mymk^{scKO}). (2E) Schematic showing experimental design to delete Mymk just before NBPI and assess myonuclear numbers and contracture pathology at P33. (2F) Single myofiber images from contralateral and NBPI biceps of control and Mymk^{scKO} mice. DAPI shows myonuclei. (2G) Quantification of nuclei per myofiber in the various groups of mice. (n=4 for control and Mymk^{scKO}). (H) Brachialis sarcomere length, where increased sarcomere length indicates reduced functional muscle length (sarcomeres in series). Reduction of myonuclear numbers by 75% in Mymk^{scKO} muscle does not impact muscle length (control, n=6 and Mymk^{scKO}, n=9). (I) Assessment of elbow extension in the various groups of mice, where 170-180° represents full range of motion. NBPI causes reduced range of motion, but reduction of myonuclear numbers in Mymk^{scKO} does not reduce range of motion in contralateral limbs or exacerbate the reduction caused by NBPI (control, n=6 and Mymk^{scKO}, n=9). Data are presented as mean±SD. Statistical analysis performed with unpaired two-tailed Student's t-tests in (2A), (2C), (2D); and with unpaired, two-tailed Student's t-test between groups and paired, two-tailed Student's t-tests between limbs of mice in each group in (2G), (2G), and (2I); except comparisons including NBPI brachialis sarcomere length in Mymk^{scKO} mice in (2H), where nonparametric tests (Mann-Whitney U test between groups and Wilcoxon signed rank test between sides) were used due to non-normal distribution of these data. *P<0.05, **P<0.01, ***P<0.001, ****P<0.0001. Scale bars, 100 μm.

FIGS. 3A-3G depict elevated protein degradation in NBPI muscle. (3A) Gene ontology analysis of the 336 genes up-regulated in muscle 2 weeks (w) after NBPI. (3B) Analysis of protein synthesis in muscle after mice were treated with puromycin, which is incorporated into nascent polypeptides. Shown is a representative puromycin western blot of muscle samples from various NBPI time points. Coomassie is used as a loading control. (3C) Quantification of the puromycin signal in NBPI muscle in (3B) expressed as a percentage of the contralateral (week 0, 1 n=5, week 2 n=6, week 3 n=3, week 4 n=3). (3D) Representative western blot for K48 ubiquitin where Coomassie is shown as a loading control. (3E) K48 ubiquitin signal intensity at multiple weeks after NBPI, expressed as a percentage of the contralateral (week 0, 1 n=5, week 2 n=6, week 3 n=5, week 4 n=6). (3F) MuRF1 transcript levels 2 weeks after NBPI (contralateral n=6, 2w NBPI n=6). (3G) Fluorescent-based assay for 20S proteasome activity, normalized to amount of protein (contralateral n=6, 2w NBPI n=6). Data are presented as mean±SD. Statistical analysis performed with paired, two-tailed Student's t-tests comparing NBPI muscle to contralateral. *P<0.05, **P<0.01, ***P<0.001, ****P<0.0001.

FIGS. 4A-4G depicts pharmacologic inhibition of the proteasome preserves longitudinal muscle growth and prevents contractures. (4A) Experimental scheme for NBPI and Bortezomib treatment. (4B) Images of forelimbs showing contractures in elbow (top) and shoulder (bottom) after NBPI, which are corrected with Bortezomib. (4C) Quantification of contracture severity, calculated as the difference in extension (elbow) or rotation (shoulder) between NBPI and contralateral. Saline and [Gly14]-HN were used as controls (saline n=9, [Gly14]-HN n=10, 0.4 mg/kg Bortezomib n=11). (4D) Schematic showing the optimized Bortezomib treatment strategy. (4E) Forelimb images showing the lack of contractures after NBPI in mice treated with 0.3 mg/kg Bortezomib beginning at P8. (4F) Contracture severity in the elbow and shoulder from the mice shown in (4E). The dotted line represents the severity in saline controls (from (4C)) (elbow n=15, shoulder n=16). (4G) Sarcomere length in the brachialis shows that 0.3 mg/kg Bortezomib preserves muscle length (saline n=8, [Gly14]-HN n=10, 0.3 mg/kg Bortezomib n=15). Data are presented as mean±SD. Statistical analysis performed with an unpaired, two-tailed Student's t-tests in (4C) and (4F); and with unpaired, two-tailed Student's t-test between groups and paired, two-tailed Student's t-tests between limbs of mice in each group in (4G). ***P<0.001, ****P<0.0001.

FIG. 5 depicts genetic evidence for myonuclear accretion after NBPI. (A) Schematic showing use of Pax7^{CreER}; Rosa26^{LacZ} mice to label MuSCs at postnatal day 7 and track their incorporation into the myofiber. (B) Representative images (left) of X-gal stained contralateral and NBPI muscle. Quantification (right) of the percentage of LacZ+ myofibers. Data are presented as mean±SD. Statistical analysis performed with a paired, two-tailed Student's t-test. ***P<0.001. Scale bar, 50 μm.

FIG. 6. Actin and myosin proteins are increased in NBPI muscle. (A) Representative western blots probed for skeletal muscle actin, fast myosin (myh1), and slow myosin (myh7) from muscle lysates at various time points after NBPI. Coomassie was used as a loading control. (B) Quantification of the signal intensity for skeletal muscle actin (weeks 0, 1, 3, 4 n=5, week 2 n=6), fast myosin (weeks 0, 1, 4 n=5, week 2 n=6, week 3 n=4), slow myosin (weeks 0, 3 n=5, week 1 n=4, weeks 2, 4 n=6). The signal intensity in NBPI muscle is expressed as a percentage of the contralateral. Data are presented as mean±SD. Statistical analysis performed with paired, two-tailed Student's t-tests comparing NBPI muscle to contralateral, except Wilcoxon Signed Rank test used for slow myosin at NBPI week 3, due to non-normally distributed data at this time point. *P<0.05, **P<0.01, ***P<0.001, ****P<0.0001.

FIGS. 7A-7E. Optimization of Bortezomib dose and timing. (7A) Survival of mice treated with saline, [Gly14]-HN, or 0.4 mg/kg Bortezomib from P0-P33 (saline n=9, [Gly14]-HN n=10, 0.4 mg/kg Bortezomib n=11). (7B) Experimental scheme to vary the timing and dose of Bortezomib. (7C) Percent of surviving mice during the various Bortezomib treatment regimens. (7D) Severity of elbow (top) and shoulder (bottom) contractures after NBPI and treatment with Bortezomib. The black dotted line is the average contracture severity from saline-treated animals and green dotted line is the average contracture severity from mice treated with 0.4 mg/kg Bortezomib from P5-P33 (from FIG. 4C). Sample sizes for (7C, 7D) are 0.2 mg/kg P5-P33 n=19, 0.3 mg/kg P5-P33 n=10, 0.4 mg/kg P8-P33 n=12, 0.4 mg/kg P12-P33 n=19. (7E) Survival curve for the mice treated with 0.3 mg/kg Bortezomib from P8-P33 (n=16). Data are presented as mean±SD. Statistical analyses were performed with unpaired, two-tailed Student's t-tests comparing each treatment group to saline controls (from FIG. 4C), except Bortezomib 0.04 mg/kg P12-P33 where Mann-Whitney U-test was used due to non-normally distributed data at this time point. *P<0.05, ****P<0.0001.

FIG. 8 depicts elbow and shoulder contracture in response to saline, [Gly14]-HN, and bortezomib+[Gly14]-HN.

FIG. 9 depicts percent survival and body weight versus days post-surgery, in saline and bortezomib+[Gly14]-HN treated animals.

FIG. 10A-10C depict elbow contracture severity, shoulder contracture severity, and survival at varying concentrations of bortezomib.

FIG. 11A-11C depict elbow contracture severity, shoulder contracture severity, and survival at bortezomib administered over various time periods.

FIG. 12A-12C depict elbow contracture severity, shoulder contracture severity, survival, and body weight at varying concentrations of bortezomib at various time periods.

FIG. 13 depicts elbow contracture severity, shoulder contracture severity, and sarcomere length at varying concentrations of bortezomib at various time periods.

FIG. 14 depicts elbow contracture severity, shoulder contracture severity, survival, and sarcomere length in response to bortezomib at various time periods.

FIG. 15 depicts body weight post surgery, saline versus Carfilzomib (“CFZ”).

FIG. 16 depicts percent survival, saline versus Carfilzomib (“CFZ”).

FIG. 17 depicts elbow contracture severity in saline, Carfilzomib (“CFZ”), and bortezomib.

FIG. 18 depicts shoulder contracture severity in saline, Carfilzomib (“CFZ”), and bortezomib.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The active agent may form salts, which are also within the scope of the preferred embodiments. Reference to a compound of the active agent herein is understood to include reference to salts thereof, unless otherwise indicated. The term “salt(s)”, as employed herein, denotes acidic and/or basic salts formed with inorganic and/or organic acids and bases. In addition, when an active agent contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps, which may be employed during preparation. Salts of the compounds of the active agent may be formed, for example, by reacting a compound of the active agent with an amount of acid or base, such as an equivalent amount, in a medium such as one in which the salt precipitates or in an aqueous medium followed by lyophilization. When the compounds are in the forms of salts, they may comprise pharmaceutically acceptable salts. Such salts may include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

Disclosed herein is a method of treating a muscle contracture in an individual in need thereof, which may comprise administration of one or more proteasome inhibitors as described herein, to said individual. In one aspect, the administration may yield an improvement in longitudinal muscle growth. For example, the administration may result in at least 80%, or at least 85%, or at least 90% or at least 95% rescue of muscle length as compared to expected muscle length in an individual that does not have a neuromuscular disorder that results in muscle contracture. In a further aspect, the administration may result in one or more measures of improvement of longitudinal muscle growth. Improvement of longitudinal muscle growth may be determined by an outcome selected from one or more of increased longitudinal muscle growth in said individual, normalized longitudinal growth in said individual wherein normalized longitudinal muscle growth means an improvement that causes the growth of said muscle to be within one standard deviation of that of a normal, healthy control individual that does not have a neuromuscular disorder, decreased impairment of longitudinal muscle growth, decreased protein degradation in longitudinal muscle, restoration or increased muscle length, an increase in brachialis length, as evidenced by a reduction in sarcomere elongation, and a preservation of length of denervated muscle.

In one aspect, the muscle contracture may be associated with a neuromuscular disorder selected from neonatal brachial plexus injury (NBPI) and cerebral palsy (CP). In one aspect, the individual may be diagnosed with cerebral palsy and the muscle contracture may be characterized by an upper neurologic lesion. In one aspect, the individual may be diagnosed with neonatal brachial plexus injury, and the muscle contracture is characterized by an upper neurologic lesion. In one aspect, the administration may result in a decrease in contracture severity in the individual.

In one aspect, the administration may result in reduction of contracture severity in the joints of the upper extremities, the lower extremities, or combinations thereof, in the treated individual. For example, the reduction of contracture severity may occur in a region selected from one or more of shoulder, elbow, and leg. Contractures of the lower extremities commonly occur in CP, while contractures of the upper extremities are a feature of brachial plexus injury. In one aspect, the administration may result in increased range of motion in a joint of the individual as compared to the range of motion prior to the administration of the proteasome inhibitor.

In one aspect, the proteasome inhibitor may be selected from a 20S proteasome inhibitor, a 26S proteasome inhibitor, or a combination thereof. In one aspect, the proteasome inhibitor may be a peptide boronates, such as, for example, Bortezomib (Velcade®) or CEP-188770, or combinations thereof.

In one aspect, the proteasome inhibitor may be co-administered with a neuroprotective agent. The neuroprotective agent may be, for example, humanin, a humanin analogue, and combinations thereof. In one aspect, the neuroprotective agent may be S14G-humanin (i.e., [Gly 14]-Humanin, as described in Gao et al., “Humanin analogue, S14G-humanin, has neuroprotective effects against oxygen glucose deprivation/reoxygenation by reactivating Jak2/Stat3 signaling through the PI3K/AKT pathway.” Exp Ther Med. 2017 October; 14 (4):3926-3934. doi: 10.3892/etm.2017.4934. Epub 2017 Aug. 16. PubMed PMID: 29043002; PubMed Central PMCID: PMC5639330, or that described in U.S. Pat. No. 9,034,825 or US 20180353570.

In one aspect, the administration may occur during a period of neonatal muscle growth of said individual. In one aspect, the administration step may occur at an age selected from less than 10 weeks of age, less than 9 weeks of age, less than 8 weeks of age, less than 7 weeks of age, less than 6 weeks of age, less than 5 weeks of age, less than 4 weeks of age, less than 3 weeks of age, less than 2 weeks of age, or less than 1 week of age.

In one aspect, the administration may be carried out at an interval selected from three times a day, twice a day, once a day, once every other day, once every two days, once every three days, once every four days, once every five days, once every six days, once a week, once every two weeks.

In one aspect, the administration step may be carried out prior to contracture development, wherein said individual exhibits one or more signs selected from paralysis or weakness of muscles during the neonatal period.

In one aspect, the method may comprise improving longitudinal muscle length in an individual in need thereof, for example, in an individual having cerebral palsy or neonatal brachial plexus injury, comprising administering to the individual a therapeutic dose of one or more proteasome inhibitors, which may include, for example, bortexomib. The administration may be limited to a period of time, for example, a time period selected from less than 12 weeks, or less than 11 weeks, or less than 10 weeks, or less than nine weeks, or less than eight weeks, or less than seven weeks, or less than six weeks, or less than five weeks, or less than four weeks, or less than three weeks, or less than two weeks, or less than one week. The period of time may be a period of time during which the individual is undergoing longitudinal muscle growth.

### Proteasome Inhibitors

The proteasome, (also referred to as multicatalytic protease (MCP), multicatalytic proteinase, multicatalytic proteinase complex, multicatalytic endopeptidase complex, 20S, 26S, or ingensin) is a large, multiprotein complex present in both the cytoplasm and the nucleus of all eukaryotic cells. It is a highly conserved cellular structure that is responsible for the ATP-dependent proteolysis of most cellular proteins (Tanaka, Biochem Biophy. Res. Commun., 1998, 247, 537). The 26S proteasome consists of a 20S core catalytic complex that is capped at each end by a 19S regulatory subunit. The archaebacterial 20S proteasome contains fourteen copies of two distinct types of subunits, α and β, which form a cylindrical structure consisting of four stacked rings. The top and bottom rings contain seven α-subunits each, while the inner rings contain seven β-subunits. The more complex eukaryotic 20S proteasome is composed of about 15 distinct 20-30 kDa subunits and is characterized by three major activities with respect to peptide substrates.

The term “proteasome inhibitor” as used herein refers to compounds which directly or indirectly perturb, disrupt, block, modulate or inhibit the action of proteasomes (large protein complexes that are involved in the turnover of other cellular proteins). The term also embraces the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above. Proteasomes control the half-life of many short-lived biological processes. At the plasma membrane of skeletal muscle fibers, dystrophin associates with a multimeric protein complex, termed the dystrophin-glycoprotein complex (DGC). Protein members of this complex are normally absent or greatly reduced in dystrophin-deficient skeletal muscle fibers and inhibition of the proteasomal degradation pathway rescues the expression and subcellular localization of dystrophin-associated proteins.

Several classes of proteasome inhibitors are known, and inhibitors of the proteolytic activity of the proteasome have been reported and are described in, for example, U.S. Pat. No. 7,223,745. Classes of proteasome inhibitors that may be used with the methods of the instant disclosure, include, for example, actives from the following classes of agents: peptide boronates, peptide aldehydes, peptide vinyl sulfones, β lactone inhibitors (e.g. lactacystin, MLN 519, NPI-0052, Marizomib (NPI-0052; salinosporamide A, described in, for example, Potts, B C et al. “Marizomib, a proteasome inhibitor for all seasons: preclinical profile and a framework for clinical trials.” Current cancer drug targets vol. 11, 3 (2011): 254-84), compounds which create dithiocarbamate complexes with metals (Disulfuram, a drug which is also used for the treatment of chronic alcoholism), and certain antioxidants (e.g. Epigallocatechin-3-gallate and catechin-3-gallate).

The class of the peptide boronates includes bortezomib (INN, PS-341; Velcade®), a compound approved in the U.S. for the treatment of relapsed multiple myeloma. See, e.g., US2009/0131367, also referred to as ([1R)-3-methyl-1-[[(2S)-1-oxo-3-phenyl-2-[(pyrazinylcarbonyl)amino]propyl]amino]butyl]-boronic acid). Bortezimib is commercially available from Millennium Pharmaceuticals Inc under the trade name Velcade, or may be prepared as described in PCT patent specification No. WO 96/13266, or by processes analogous thereto. Bortezimib specifically interacts with a key amino acid, namely threonine, within the catalytic site of the proteasome. Another peptide boronate is CEP-18770.

Peptide aldehydes have been reported to inhibit the chymotrypsin-like activity associated with the proteasome and may be used as a proteasome inhibitor. Dipeptidyl aldehyde inhibitors that have IC50 values in the 10-100 nM range in vitro have also been reported. A series of similarly potent in vitro inhibitors from α-ketocarbonyl and boronic ester derived dipeptides has also been reported (U.S. Pat. Nos. 5,614,649; 5,830,870; 5,990,083; 6,096,778; 6,310,057; U.S. Pat. App. Pub. No. 2001/0012854, and WO 99/30707).

Further exemplary proteasome inhibitors may be selected from, one or more of the following:(benzyloxycarbonyl)-Leu-Leu-phenylalaninal, 2,3,5a,6-tetrahydro-6-hydroxy-3-(hydroxymethyl)-2-methyl-10H-3α, 10a-epidithio-pyrazino[1,2a]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx3L3VS, AdaLys(Bio)Ahx3L3VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-β-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-β-lactone, mevinolin, MK-803, NIP-L3VS, NP-LLL-VS, NPI-0052 (salinosporamide A), MLN519 (PS-519), NLVS (trileucine vinyl-sulfone), ritonavir, Ro6-9920, Z-LLF-CHO, Z-LL-B(OH)2, RRRPRPPYLPR, Tyropeptin A, ZL3VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132 [Z-Leu-Leu-Leu-CHO] (a proteasome and NF-κB inhibitor), MG-262, MG-115 (CBZ-leucyl-leucyl-norvalinal) and ALLN (N-acetyl-leucyl-leucyl-norleucinal) (see also, U.S. Pat. No. 8,501,713, which describes these classes of proteasome inhibitors), α-methylomuralide, MG-101, peptide epoxyketones (e.g. epoxomicin, PR-171 (carfilzomib, “CFZ”)), omuralide, lactacystin (a Streptomyces metabolite that specifically inhibits the proteolytic activity of the proteasome complex, which is capable of inhibiting the proliferation of several cell types), NEOSH101, N-terminal peptidyl boronic ester and acid compounds (U.S. Pat. Nos. 4,499,082 and 4,537,773; WO 91/13904; Kettner, et al, which have been reported to be inhibitors of certain proteolytic enzymes).

In one aspect, the proteasome inhibitor may be carfilzomib, or “CFZ.” CFZ is a novel irreversible proteasome inhibitor that is structurally and mechanistically different from BTZ and is now FDA-approved for treatment of relapsed/refractory MM. CFZ selectively inhibits the chymotrypsin-like activity of both the constitutive proteasome and the immunoproteasome.

In one aspect, the proteasome inhibitor may inhibit the peptidase activities of the proteasome, for example, a proteasome inhibitor as reported in U.S. patent application Ser. No. 08/212,909, filed Mar. 15, 1994, Palombella, et al., WO 95/25533, WO 94/17816, Stein, et al., U.S. Pat. No. 5,693,617, indanone derivatives as described in Lum et al., U.S. Pat. No. 5,834,487, alpha-ketoamide compounds as described in Wang et al., U.S. Pat. No. 6,075,150, 2,4-diamino-3-hydroxycarboxylic acid derivatives as proteasome inhibitors as described in France, et al., WO 00/64863, carboxylic acid derivatives as proteasome inhibitors as reported by Yamaguchi et al., EP 1166781, bivalent inhibitors of the proteasome as reported in Ditzel, et al., EP 0 995 757, and 2-Aminobenzylstatine derivatives that inhibit non-covalently the chymotrypsin-like activity of the 20S proteasome.

Some further proteasome inhibitors can contain boron moieties. For example, Drexler et al., WO 00/64467, report a method of selectively inducing apoptosis in activated endothelial cells or leukemic cells having a high expression level of c-myc by using tetrapeptidic boronate containing proteasome inhibitors. Furet et al., WO 02/096933 report 2-[[N-(2-amino-3-(heteroaryl or aryl)propionyl)aminoacyl]amino]-alkylboronic acids and esters for the therapeutic treatment of proliferative diseases in warm-blooded animals. U.S. Pat. Nos. 6,083,903; 6,297,217; 5,780,454; 6,066,730; 6,297,217; 6,548,668; U.S. Patent Application Pub. No. 2002/0173488; and WO 96/13266 report boronic ester and acid compounds and a method for reducing the rate of degradation of proteins. Pharmaceutically acceptable compositions of boronic acids and novel boronic acid anhydrides and boronate ester compounds are reported by Plamondon, et al., U.S. Patent Application Pub. No. 2002/0188100. A series of di- and tripeptidyl boronic acids are shown to be inhibitors of 20S and 26S proteasome in Gardner, et al., Biochem. J., 2000, 346, 447. Other boron-containing peptidyl and related compounds are reported in U.S. Pat. Nos. 5,250,720; 5,242,904; 5,187,157; 5,159,060; 5,106,948; 4,963,655; 4,499,082; and WO 89/09225, WO/98/17679, WO 98/22496, WO 00/66557, WO 02/059130, WO 03/15706, WO 96/12499, WO 95/20603, WO 95/09838, WO 94/25051, WO 94/25049, WO 94/04653, WO 02/08187, EP 632026, and EP 354522.

20S Proteasome inhibitors may include, for example Aclacinomycin A (a non-peptidic inhibitor of CTRL and Calpain), Withaferin A (a potent inhibitor of angiogenesis, a vimentin and proteasome inhibitor, Simvastatin (an HMGCR inhibitor and anti-proliferative agent, Epoxomicin (a potent chymotrypsin-like proteasome inhibitor (CTRL)), Gliotoxin (a toxic epipolythiodioxopiperazine metabolite that induces apoptosis and inhibits NF-κB), clasto-Lactacystin beta-Lactone (a 20S proteasome and cathepsin A inhibitor), Bortezomib, AdaAhx3L3VS (an irreversible inhibitor of chymotrypsin-like, trypsin-like, and PGPH activities of the 20S proteasome), MG-115 (a compound that inhibits the chymotrypsin-like activity of the proteasome), Proteasome Inhibitor VIII, beta-Lactam 3 (a selective, irreversible inhibitor of the 20S proteasome), 8-Hydroxyquinoline hemisulfate salt hemihydrate (a 20S proteasome inhibitor, Lactacystin (a proteasome inhibitor and cathepsin A inhibitor), all available from Santa Cruz Biotechnology.

In a further aspect, the proteasome inhibitor may be a 26S proteasome inhibitor, which may include Bortezomib MG-115, (a compound that inhibits the chymotrypsin-like activity of the proteasome), Proteasome Inhibitor I (a selective inhibitor of chymotrypsin-like activities in the 26S proteasome (MCP)), all available from Santa Cruz Biotechnology, and PS-341, a 26S Proteasome Inhibitor available from R&D systems at www.rndsystems.com.

In a yet further aspect, the disease states disclosed herein may be treated by administration of a MuRF1 inhibitor, such as that described in, for example, Bowen et al., “Small-molecule inhibition of MuRF1 attenuates skeletal muscle atrophy and dysfunction in cardiac cachexia,” J Cachexia Sarcopenia Muscle. 2017 December; 8 (6):939-953. doi: 10.1002/jcsm.12233. Epub 2017 Sep. 8; Eddins et al., Targeting the ubiquitin E3 ligase MuRF1 to inhibit muscle atrophy. Cell Biochem Biophys. 2011 June; 60 (1-2):113-8. doi: 10.1007/s12013-011-9175-7; or Bowen, T. S., Adams, V., Werner, S., Fischer, T., Vinke, P., Brogger, M. N., . . . Labeit, S. (2017). Small-molecule inhibition of MuRF1 attenuates skeletal muscle atrophy and dysfunction in cardiac cachexia. Journal of cachexia, sarcopenia and muscle, 8 (6), 939-953. doi:10.1002/jcsm.12233.

In one aspect, the agent comprises bortezomib, and may be administered at a dose of about 0.05 mg/kg to about 5 mg/kg, or from about 0.1 mg/kg to about 4 mg/kg, or from about 0.2 mg/kg to about 3 mg/kg, or from about 0.3 to about 2 mg/kg, or from about 0.5 to about 1 mg/kg. In certain aspects, the initial dose may be delayed until the individual is at least one week of age, or at least two weeks of age, or at least three weeks of age, or at least four weeks of age, or at least five weeks of age, or at least six weeks of age, or at least seven weeks of age, or at least eight weeks of age, or at least nine weeks of age, or at least ten weeks of age, or at least 11 weeks of age, or at least 12 weeks of age. In one aspect, the dose is escalated as the age of the individual increases. For example, an individual may be administered 0.5 mg/kg at one week of age, and at two weeks of age, the dose may be increased by 0.1 or 0.2, or 0.3, or 0.4, or 0.5, or 0.6, or 0.7, or 0.8, or 0.9, or 1.0 mg/kg over a period of time of about one week, or every two weeks, or every three weeks, or every four weeks, or every five weeks, or every six weeks, or every seven weeks, or every eight weeks.

### Pharmaceutical Compositions

In one aspect, active agents provided herein may be administered in an dosage form selected from intravenous or subcutaneous unit dosage form, oral, parenteral, intravenous, and subcutaneous. In some embodiments, active agents provided herein may be formulated into liquid preparations for, e.g., oral administration. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

In one aspect, pharmaceutical compositions may be isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, active agents provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants, such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

In some embodiments, each tablet or capsule contains from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some embodiments, tablets or capsules are provided in a range of dosages to permit divided dosages to be administered. A dosage appropriate to the patient and the number of doses to be administered daily may thus be conveniently selected. In certain embodiments two or more of the therapeutic agents may be incorporated to be administered into a single tablet or other dosage form (e.g., in a combination therapy); however, in other embodiments the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, or karaya, or alginic acid or salts thereof.

Binders may be used to form a hard tablet. Binders include materials from natural products such as acacia, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy.

The active ingredients may be prepared for pulmonary delivery in particulate form with an average particle size of from 0.1 um or less to 10 um or more, for example, from about 0.2, 0.3, 0.4, 0.5, 0.6, 0.7, 0.8, or 0.9 □m to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 □m. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used. Liposomes, microcapsules, microspheres, inclusion complexes, and other types of carriers may also be employed.

Pharmaceutical formulations suitable for use with a nebulizer, either jet or ultrasonic, typically comprise the active agent dissolved or suspended in water at a concentration of about 0.01 or less to 100 mg or more of active agent per mL of solution, for example, from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation may also include a buffer and a simple sugar (e.g., for protein stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device generally comprise a finely divided powder containing the active ingredients suspended in a propellant with the aid of a surfactant. The propellant may include conventional propellants, such as chlorofluorocarbons, hydrochlorofluorocarbons, hydrofluorocarbons, and hydrocarbons. Example propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing active agent, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, for example, from about 5, 10, 15, 20, 25, 30, 35, 40, 45, or 50 wt. % to about 55, 60, 65, 70, 75, 80, 85, or 90 wt. % of the formulation.

In some embodiments, an active agent provided herein may be administered by intravenous, parenteral, or other injection, in the form of a pyrogen-free, parenterally acceptable aqueous solution or oleaginous suspension. Suspensions may be formulated according to methods well known in the art using suitable dispersing or wetting agents and suspending agents. The preparation of acceptable aqueous solutions with suitable pH, isotonicity, stability, and the like, is within the skill in the art. In some embodiments, a pharmaceutical composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art.

The duration of the injection may be adjusted depending upon various factors, and may comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

In some embodiments, active agents provided herein may additionally employ adjunct components conventionally found in pharmaceutical compositions in their art-established fashion and at their art-established levels. Thus, for example, the compositions may contain additional compatible pharmaceutically active materials for combination therapy) or may contain materials useful in physically formulating various dosage forms, such as excipients, dyes, thickening agents, stabilizers, preservatives or antioxidants.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Cerebral palsy and neonatal brachial plexus injury are the two most common causes of neuromuscular dysfunction in childhood, occurring in a combined 1 per 200 live births¹⁻⁴. Despite differing in the type of neurologic lesion (upper vs. lower motor neuron), both conditions lead to similar muscle contractures, which dramatically reduce joint range of motion and limit the functional use of limbs for ambulating, reaching, and other activities of daily living. Furthermore, the muscular contractures alter the physical forces on the developing skeleton, leading to progressive dysplasia and dislocation of joints⁵⁻⁹. These contractures are the primary driver of the need for rehabilitative and surgical therapies, assistive devices, and accommodations for daily functioning^{10,11}. However, no existing treatment strategies alter the actual contracture pathology, and instead can worsen function by further weakening already abnormal muscles¹²⁻¹⁵. As a result, the contractures and their secondary skeletal consequences remain unchecked, leading to pain, loss of physical function, and heavy reliance on costly health care and supportive services. It is therefore imperative to gain a better understanding of contracture pathogenesis to develop novel strategies to prevent contractures.

Applicant has previously demonstrated in a mouse model of NBPI that contractures result from impaired longitudinal muscle growth. The presumed driver of neonatal muscle growth is myonuclear accretion from muscle stem cells (MuSCs), which differentiate and fuse to existing myofibers during growth. Using a mouse model of NBPI it has been demonstrated by Applicant that denervation does not prevent myonuclear accretion and that reduction of myonuclear number has no effect on muscle length or contracture development, providing definitive evidence that altered myonuclear accretion is not a driver of neuromuscular contractures. In contrast, Applicant observed increased protein degradation in NBPI muscle, and Applicant demonstrate that contractures can be pharmacologically prevented with the proteasome inhibitor, Bortezomib. These studies provide the first strategy to prevent neuromuscular contractures by correcting the underlying deficit in longitudinal muscle growth.

Applicant developed a mouse model of NBPI that causes contractures precisely mimicking the human phenotype in both NBPI and CP¹⁶. With this model, it was discovered that neuromuscular contractures result from impaired longitudinal growth of neonatally denervated muscle¹⁶⁻¹⁹ a finding that has been replicated in subsequent animal^{20,21}, clinical²²⁻²⁴, and computational analysis^{25,26} studies. Furthermore, the impaired longitudinal muscle growth following NBPI is characterized by overstretched sarcomeres identical to those seen in human muscles responsible for contractures in cerebral palsy²⁷. Applicant found in this model that contractures do not occur following muscle denervation outside the neonatal period¹⁹, consistent with the clinical observations that BPI in later childhood does not cause contractures²⁸ and suggesting a unique biologic susceptibility of neonatal longitudinal muscle growth to denervation. However, in contrast to the vast knowledge of the mechanisms that regulate muscle width, the processes in muscle that govern muscle length during the neonatal period are unexplored.

In general, muscle grows by two basic processes: (1) fusion of muscle stem cells (MuSCs)²⁹, to growing multinucleated myofibers (myonuclear accretion), and (2) an anabolic balance between protein synthesis and protein degradation within the myofibers. The contributions of these mechanisms to longitudinal muscle growth have never been experimentally dissected. A central role has been assumed for myonuclear accretion in both neonatal muscle growth and contracture development, since prior investigations have found that myonuclear accretion is unique to neonatal muscle growth^{30,31}, and because others have found MuSC depletion following longterm denervation³² or in longstanding contractures from cerebral palsy³³⁻³⁶. However, these latter findings have been based on analyses of muscles obtained after contractures have formed, so causation was not able to be determined.

Applicant found that neonatal denervation does not prevent myonuclear accretion, and that inhibiting myonuclear accretion does not impair longitudinal muscle growth. These findings rule out a role for myonuclear accretion in longitudinal muscle growth and contracture development. Furthermore, Applicant found that denervation causes elevation in both protein synthesis and protein degradation, only the latter of which could explain reduced muscle growth. Importantly, Applicant discovered that inhibition of proteasome-mediated protein degradation restores muscle length and prevents contractures following NBPI, identifying a mechanistic underpinning of contracture pathogenesis and uncovering a novel strategy to prevent neonatal neuromuscular contractures.

### Results

### Dysregulation of Muscle Stem Cells During Contracture Development

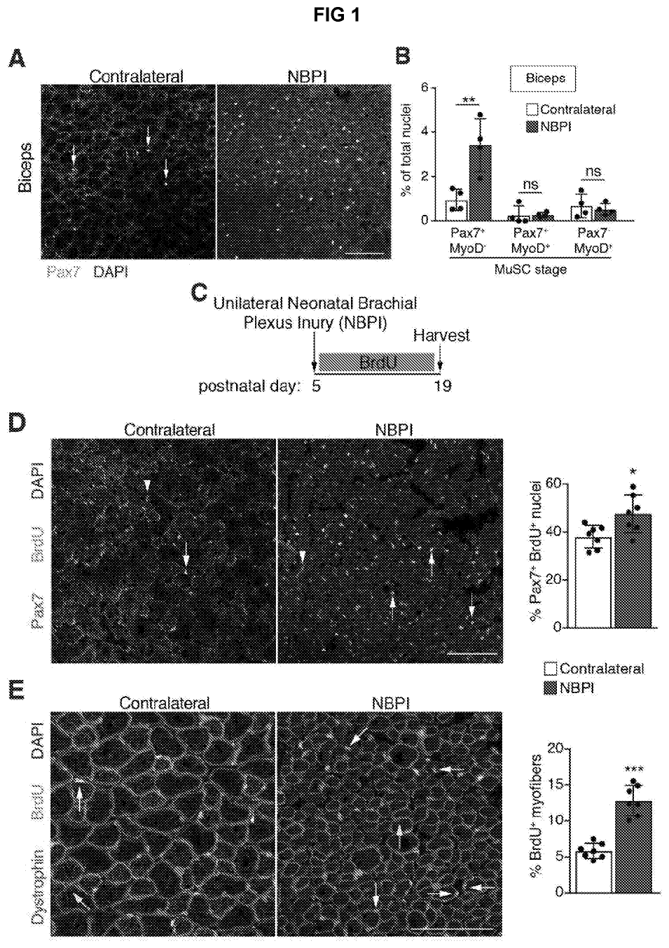

Because a unique property of neonatal muscle is the high rate of fusion between muscle progenitors and myofibers that ultimately increases myonuclear numbers³⁰, Applicant assessed whether MuSC dysregulation could contribute to contracture pathogenesis. It has been previously shown that MuSC numbers are reduced in muscle after neonatal denervation³² and in CP^{33,36}, although these analyses were performed after the time period in which contractures are established, leaving it unclear whether dysregulation of MuSCs are a cause or consequence of the pathology. Applicant thus investigated quiescent and activated MuSC populations before and during contracture development in Applicant's established murine model of NBPI, where unilateral surgical excision of the brachial plexus (nerve roots C5-T1) in postnatal (P) day 5 mice results in forelimb muscle denervation and reliably causes contractures in the shoulder and elbow consistent with the human phenotype within four weeks post-NBPI^{16,19}. Applicant first immunostained for Pax7, a marker of MuSCs, in contralateral (normally innervated) and NBPI (denervated) biceps muscles two weeks after denervation and observed elevated levels of Pax7⁺ cells in NBPI muscle (FIG. 1, A). Applicant further assessed the MuSC populations by immunostaining biceps sections with Pax7 and MyoD, a marker for activation of the myogenic program, and by quantifying the percentage of MuSCs that were Pax7⁺ MyoD⁻ (quiescent), Pax7⁺ MyoD⁺ (activated), Pax7⁻ MyoD⁺ (differentiated). Applicant found the same levels of activated and differentiated MuSCs in contralateral and NBPI muscle, but an increase in quiescent cells in NBPI muscle (FIG. 1, B), suggesting MuSC dysregulation. One possibility to explain the abundance of quiescent MuSCs is a block to activation/proliferation, which could also conceptually explain impaired muscle growth. Applicant therefore performed unilateral NBPI on P5 wild-type (WT) mice and treated them with BrdU for two weeks (FIG. 1, D). The number of Pax7⁺ cells incorporating BrdU at two weeks post-NBPI was increased compared to the contralateral muscle (FIG. 1d), ruling out a block to proliferation among MuSCs. These data together indicate that while MuSCs exhibit aberrant properties in neonatally denervated muscle, they are present and capable of proliferation and differentiation.

Still, another mechanism by which MuSC dysregulation could impact muscle length is altered myonuclear accretion, leading to myonuclear numbers that are insufficient for building sarcomeres and establishing muscle length. To assess myonuclear accretion in NBPI, Applicant used the same BrdU-labeling protocol disclosed herein (FIG. 1, C), but assessed BrdU⁺ nuclei within a dystrophin⁺ myofiber as an indicator of fusion of new nuclei, because myonuclei already within the myofiber are not proliferative and are unable to incorporate BrdU. Denervated muscle two weeks after NBPI exhibited increased percentages of myofibers containing BrdU⁺ myonuclei compared to the contralateral side (FIG. 1, E). To complement this approach, Applicant also genetically labeled MuSCs and tracked their incorporation into the myofiber by crossing the MuSC-specific tamoxifen-inducible Pax7^{CreER} mouse with a Rosa26^{LacZ} reporter. Pax7^{CreER}; Rosa26^{LacZ} mice were subjected to NBPI at P5, treated with tamoxifen at P7 and analyzed for LacZ⁺ myofibers at P19 (FIG. 5, A). X-gal staining of sections revealed LacZ⁺ myofibers in both contralateral and NBPI muscle, and quantification revealed an increased percentage of LacZ⁺ fibers two weeks after NBPI (FIG. 5, B). These data suggest that myonuclear accretion is not globally reduced in denervated muscle, and may even be increased.

### Reduced Myonuclear Accretion Does Not Impair Longitudinal Muscle Growth or Induce Contractures

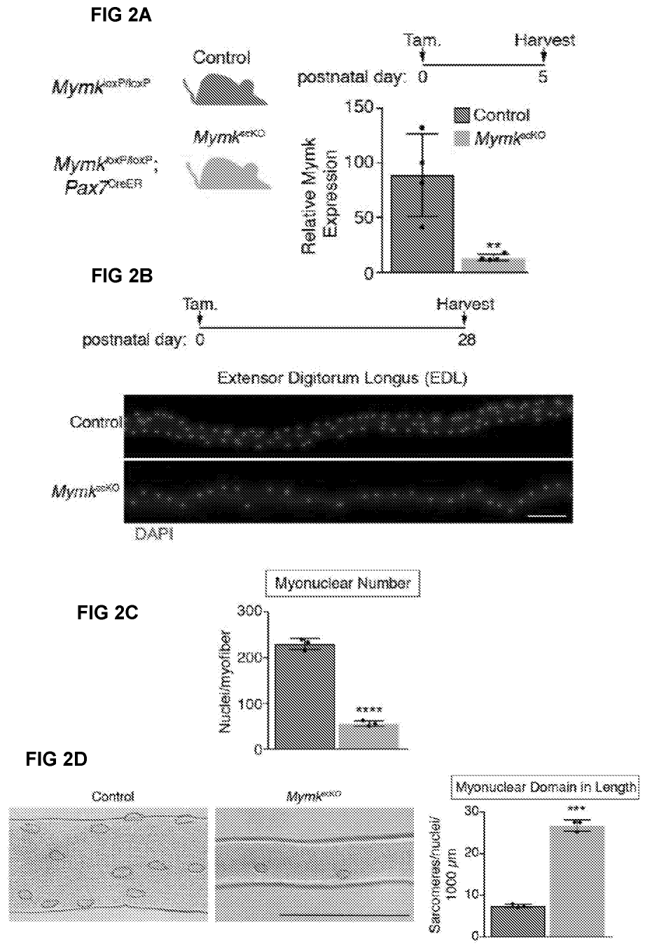

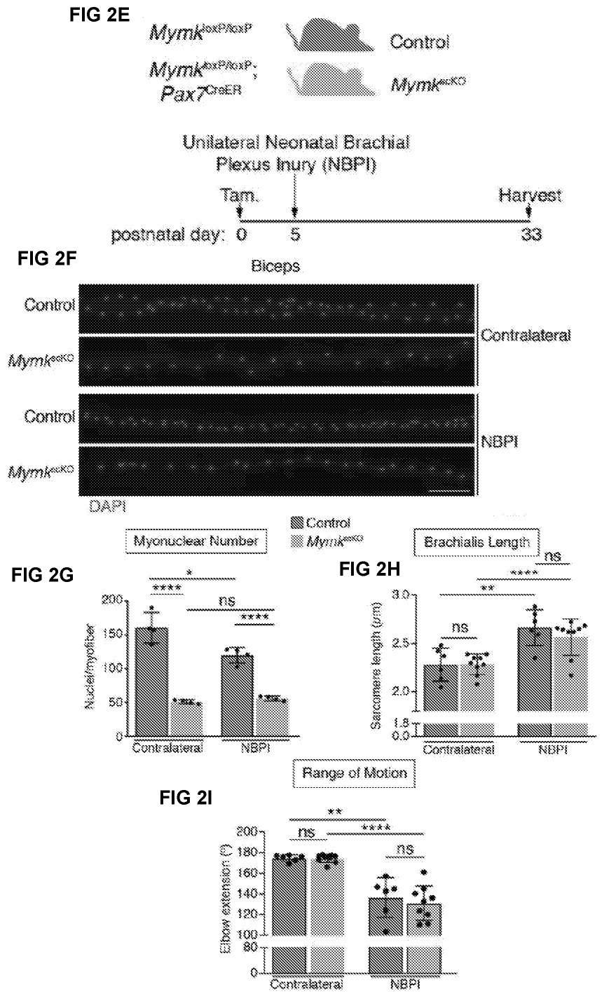

Because Applicant's findings suggesting normal or increased MuSC numbers and activity during the time frame of contracture pathogenesis are in contrast to others' findings indicating fewer MuSCs^{33,37} with less in vitro myogenic capacity³⁸ after contractures have formed, Applicant next experimentally manipulated MuSC-mediated myonuclear accretion to definitively outline the role of myonuclear accretion in longitudinal muscle growth and contractures. Applicant blocked myonuclear accretion through genetic deletion of Myomaker (Mymk), a muscle-specific protein required for muscle progenitor fusion³⁹, specifically in MuSCs during the early postnatal period. Applicant treated Mymk^{loxP/loxP} (control) and Mymk^{loxP/loxP}; Pax7^{CreER} Mymk^{scKO}) mice^{40,41} with tamoxifen at P0 and found significant down-regulation of Mymk expression in muscle at P5 (FIG. 2, A). Moreover, a 75% reduction of nuclear number in hindlimb myofibers at P28 was observed (FIG. 2, B and C), establishing that experimental manipulation of myonuclear accretion can be achieved during the time frame of contracture formation following NBPI at P5. Of note, the reduced myonuclear number in Mymk^{scKO} myofibers was characterized by an increased myonuclear domain per unit length, measured in sarcomeres per nucleus over 1000 μm segments of the myofiber (FIG. 2, D). These data indicate that sarcomere addition can occur in series without the full complement of myonuclear number.

Having established the ability to limit myonuclear accrual during the relevant developmental window, Applicant utilized the Mymk^{scKO} model to directly evaluate if reduced myonuclear numbers would cause contractures at baseline or exacerbate the NBPI phenotype. Control and Mymk^{scKO} mice were treated with tamoxifen at P0, followed by unilateral NBPI at P5, and mice were harvested at P33 (four weeks post-NBPI) (FIG. 2, E). Single myofibers from the biceps were analyzed for numbers of myonuclei, which revealed that deletion of Myomaker caused the expected reduction of nuclei per myofiber in both contralateral and NBPI muscle (FIG. 2, F and G). Applicant did observe a reduction of myonuclei in control NBPI biceps compared to control contralateral biceps, but myonuclear numbers in both Mymk^{scKO} biceps were significantly reduced compared to control NBPI muscle (FIG. 2, F and G). These data demonstrate that the Mymk^{scKO} model reduces myonuclear accretion beyond what may occur following NBPI alone.

Applicant then determined if reduction of myonuclear numbers impacts muscle length and development of contractures. Brachialis length was measured as sarcomere length at a controlled joint position, where increased sarcomere length indicates sarcomere overstretch or fewer sarcomeres in series⁴². This parameter was unchanged in contralateral (normally innervated) muscles of control and Mymk^{scKO} mice, and while NBPI resulted in increased sarcomere length (reduced muscle length) in both groups of mice, loss of Myomaker and reduction of myonuclear number did not exacerbate the pathology (FIG. 2h). Similarly, NBPI significantly reduced passive elbow extension in both groups, but Myomaker deletion did not worsen the reduction of range of motion caused by NBPI or reduce the range of motion on the contralateral side (FIG. 2, I). Thus, reducing myonuclear number does not elicit defects in muscle length or cause contractures, definitively demonstrating that myonuclear number does not control longitudinal muscle growth or NBPI-induced contractures.

### Neonatally Denervated Muscle is Characterized by Altered Protein Balance

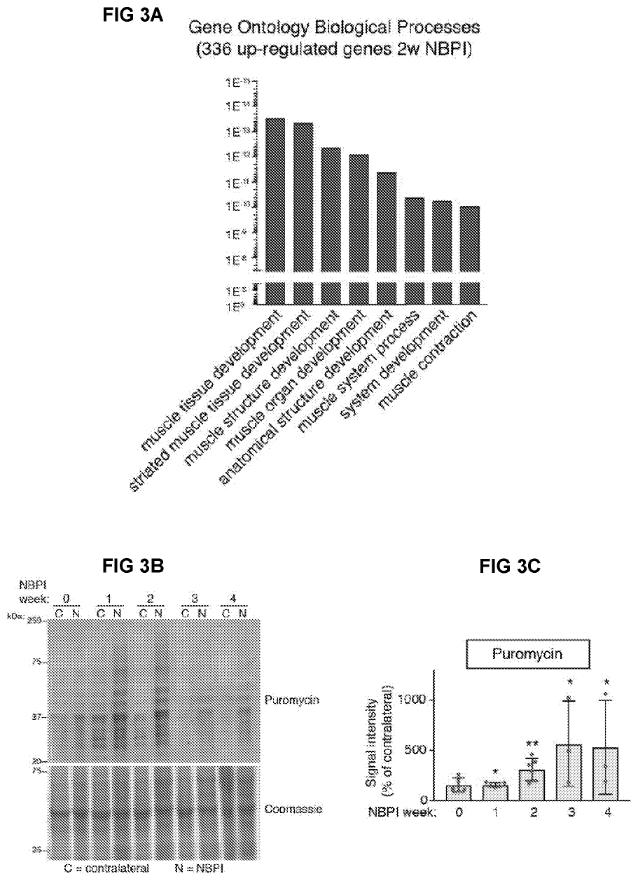

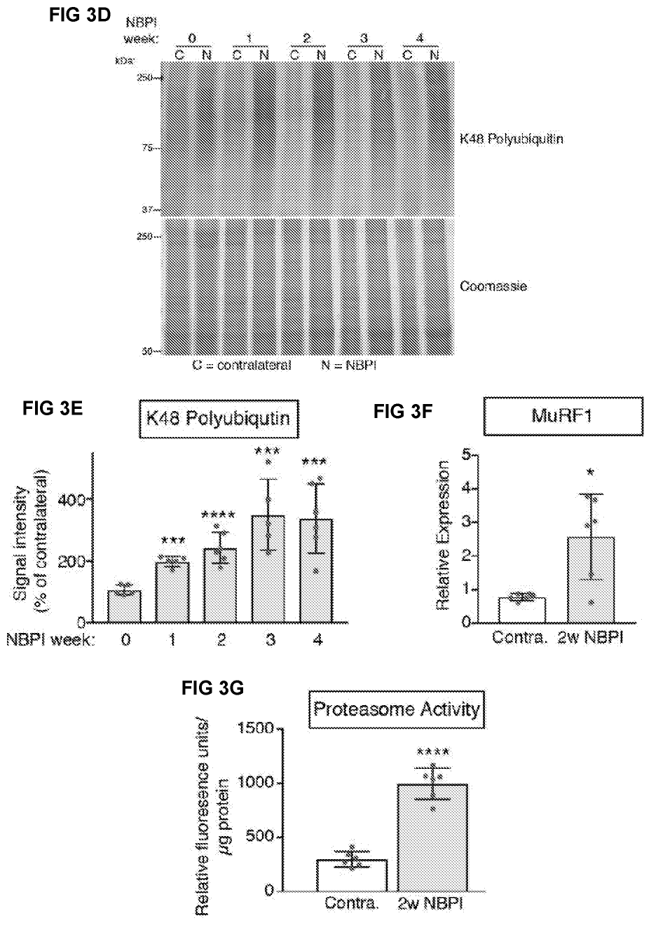

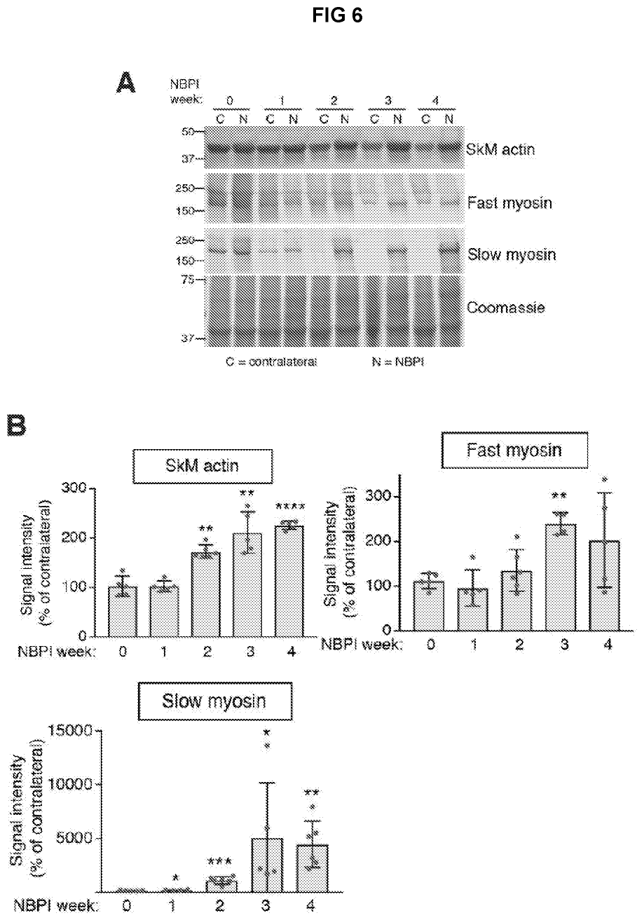

Having eliminated myonuclear accretion, or myonuclear number, as a relevant mechanism in longitudinal muscle growth and contractures, Applicant hypothesized that impaired muscle growth could be explained by reduced protein synthesis or increased protein degradation. Applicant performed RNA-sequencing on contralateral and NBPI muscle three weeks after surgery and found 336 up-regulated and 22 down-regulated genes. Gene ontology analysis revealed that denervation causes up-regulation of genes predominantly related to muscle development and structure (FIG. 3, A), suggesting that denervated muscle is transcriptionally competent. Applicant then tested if denervated muscle is able to synthesize protein at the translational level, as assessed through puromycin incorporation into nascent polypeptides, at multiple time-points post-NBPI. Applicant observed normal protein synthesis in NBPI muscle just after denervation (week 0) but an increase compared to contralateral muscle at all later time points (FIG. 3, B and C). Moreover, protein levels of skeletal muscle actin and both slow and fast myosin were elevated in denervated muscle following NBPI (FIG. 6). Thus, protein synthesis is elevated following NBPI, which conceptually cannot explain the mechanism of contracture pathology since increased protein synthesis should allow more muscle growth.

Applicant next employed multiple approaches to evaluate protein degradation, a process known to be activated in adult denervated muscle. Indeed, the ubiquitin-proteasome pathway accounts for 90% of the protein breakdown in adult denervation-induced muscle atrophy⁴³. Applicant discovered elevated K48-ubiquitinated proteins in denervated muscle at all time-points post-NBPI (FIG. 3, D and E). Additionally, in neonatally denervated muscle Applicant observed increased expression of MuRF1 (FIG. 3, F), a muscle-specific E3 ubiquitin ligase that is a central factor eliciting the cascade of protein degradation in muscle⁴⁴. Finally, using a commercially available assay for catalytic activity of the 20S proteasome⁴⁵, Applicant found increased proteasome activity in denervated muscle two weeks post-NBPI (FIG. 3, G). Overall, multiple points in the protein degradation pathway are increased following NBPI, which could explain the impaired growth of neonatally denervated muscle.

### Pharmacological Inhibition of the Proteasome Prevents Contractures

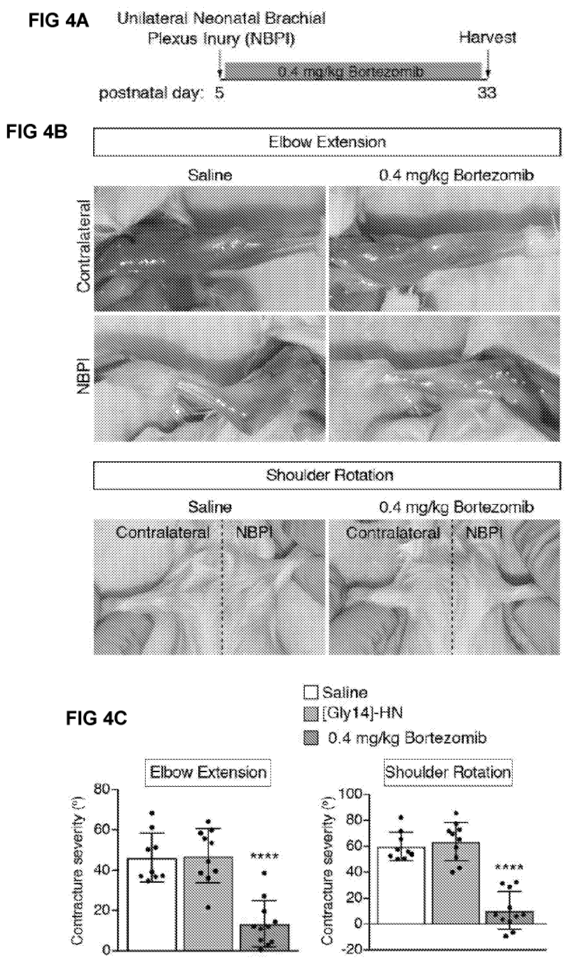

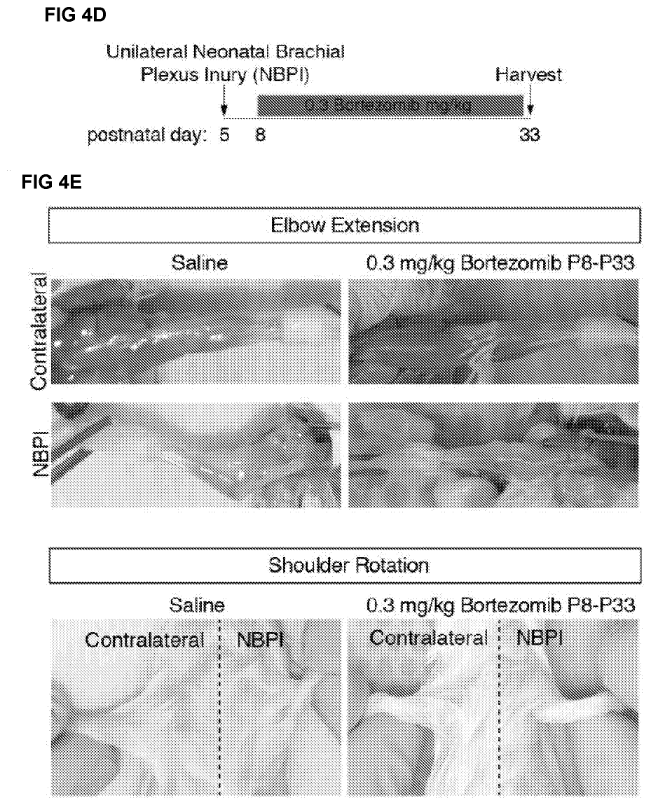

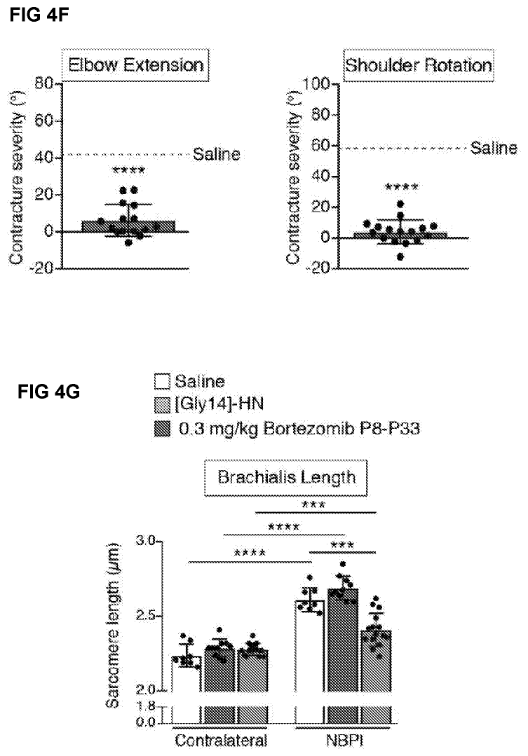

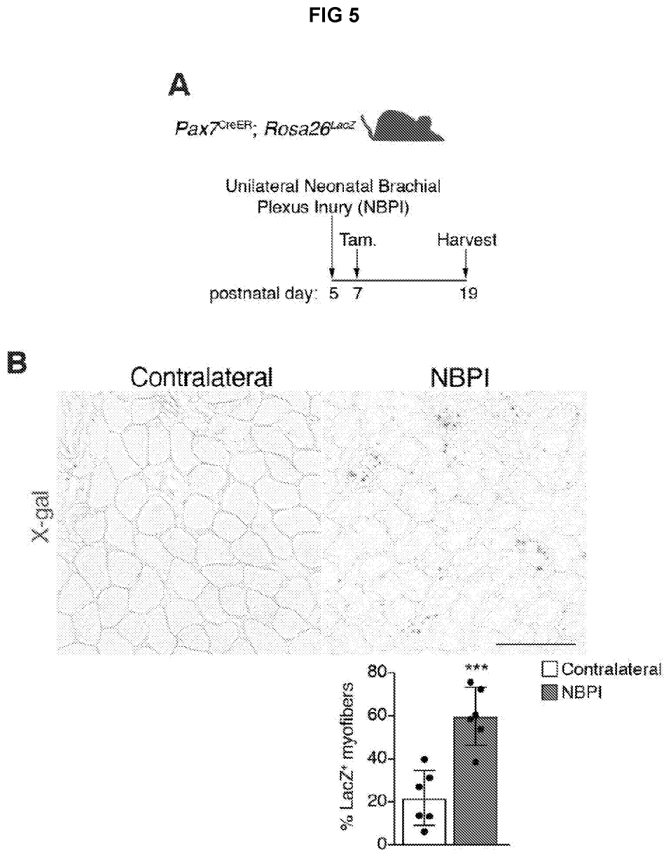

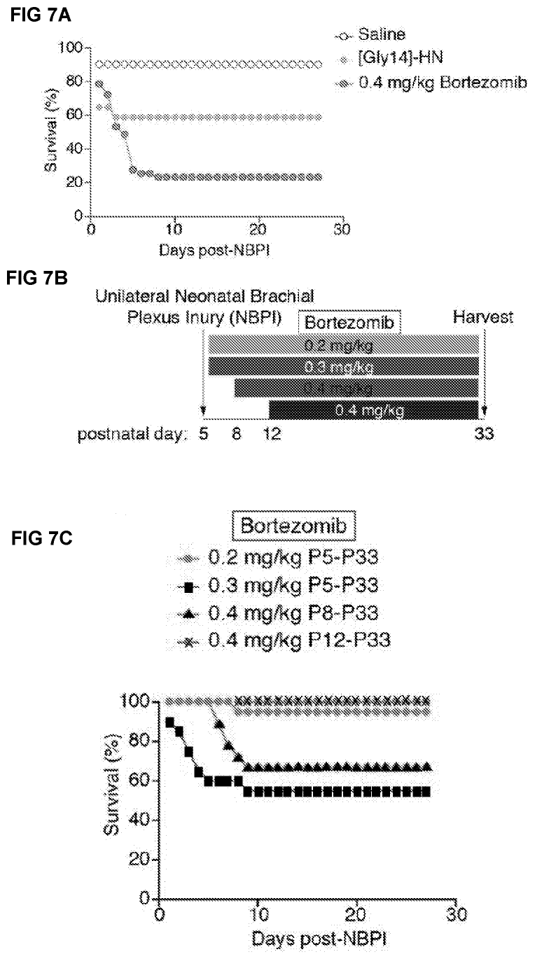

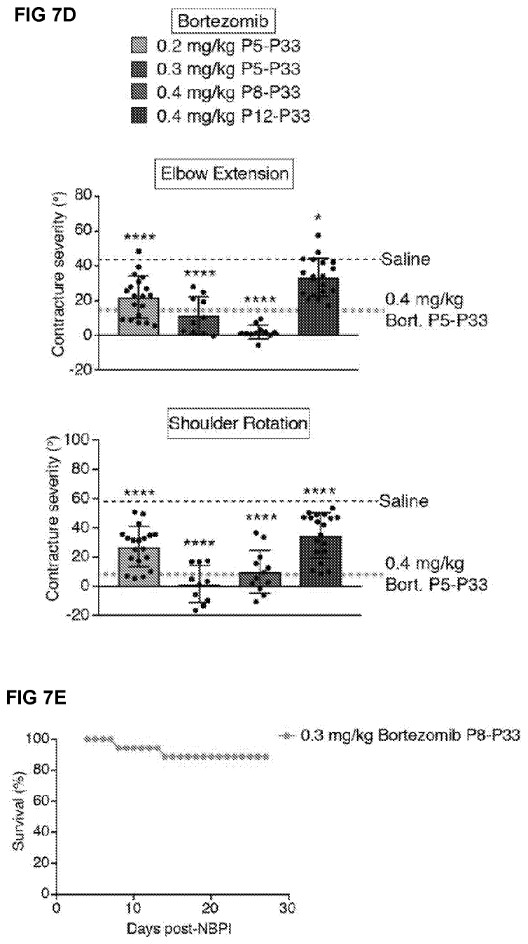

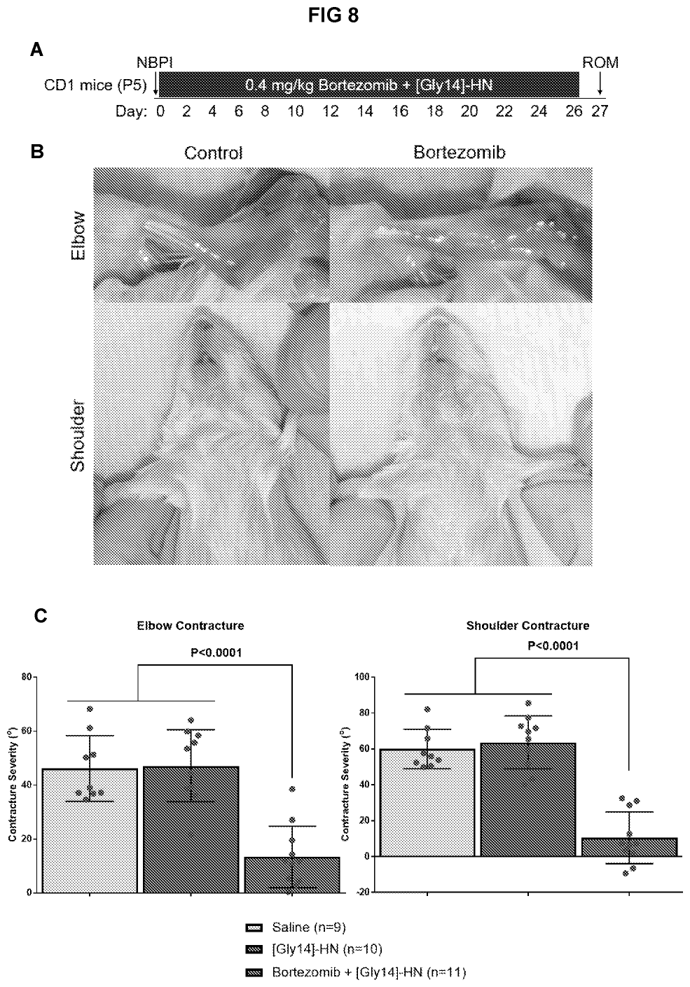

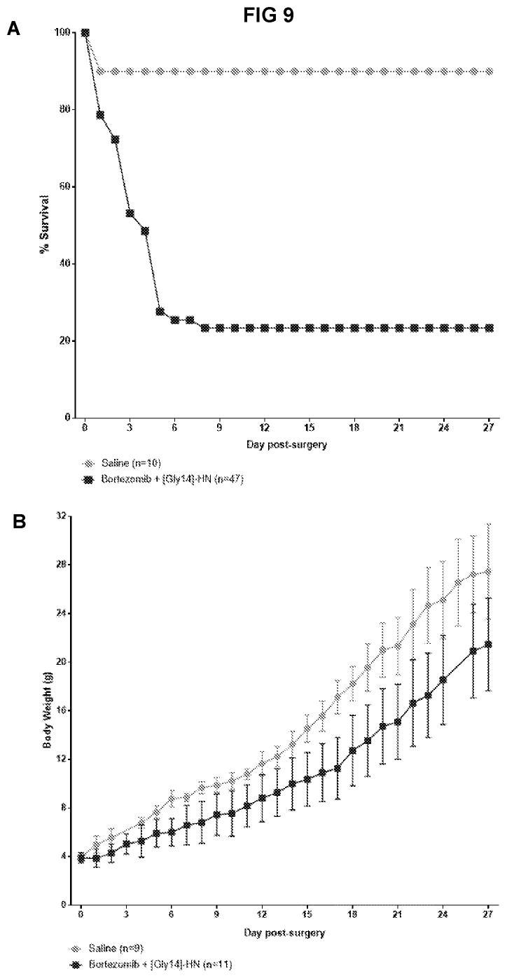

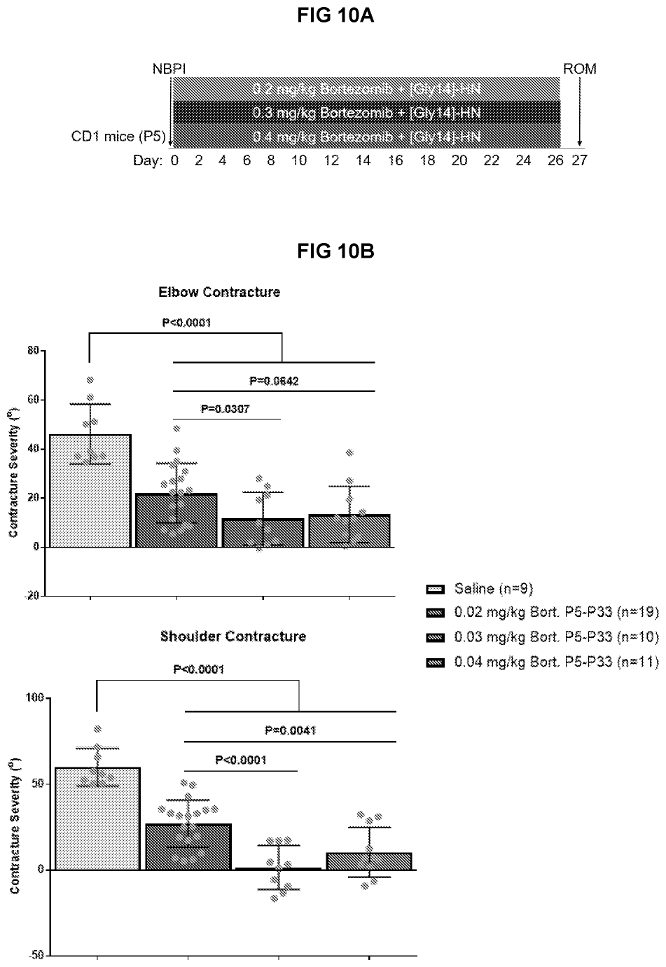

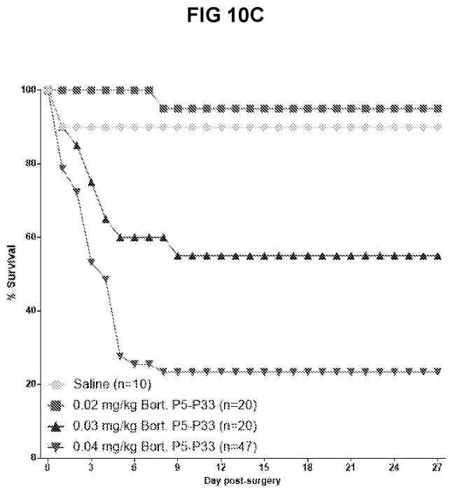

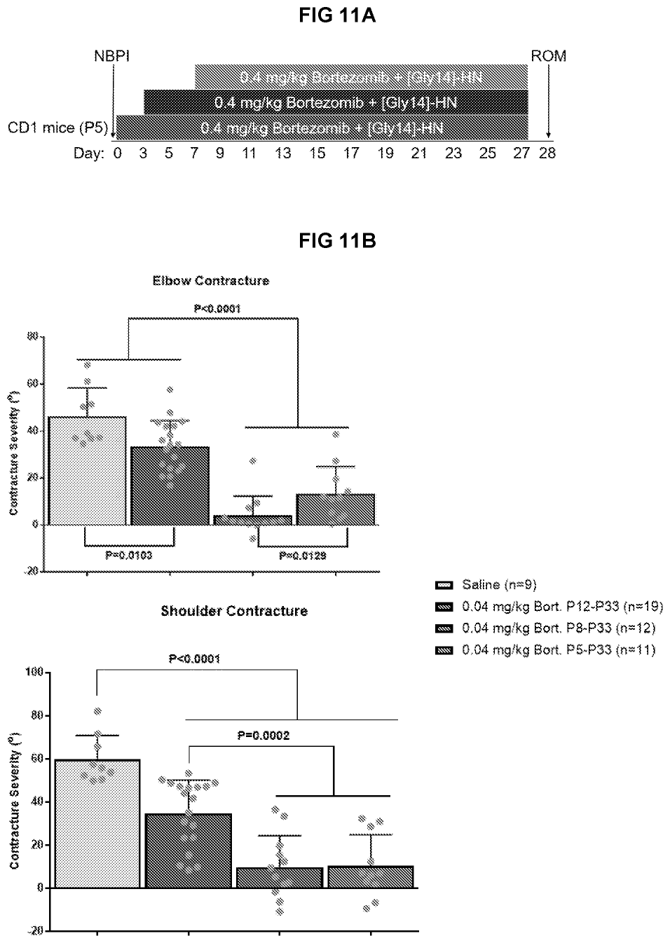

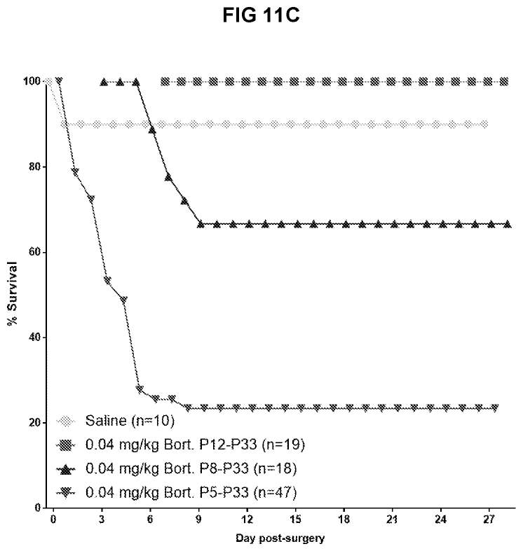

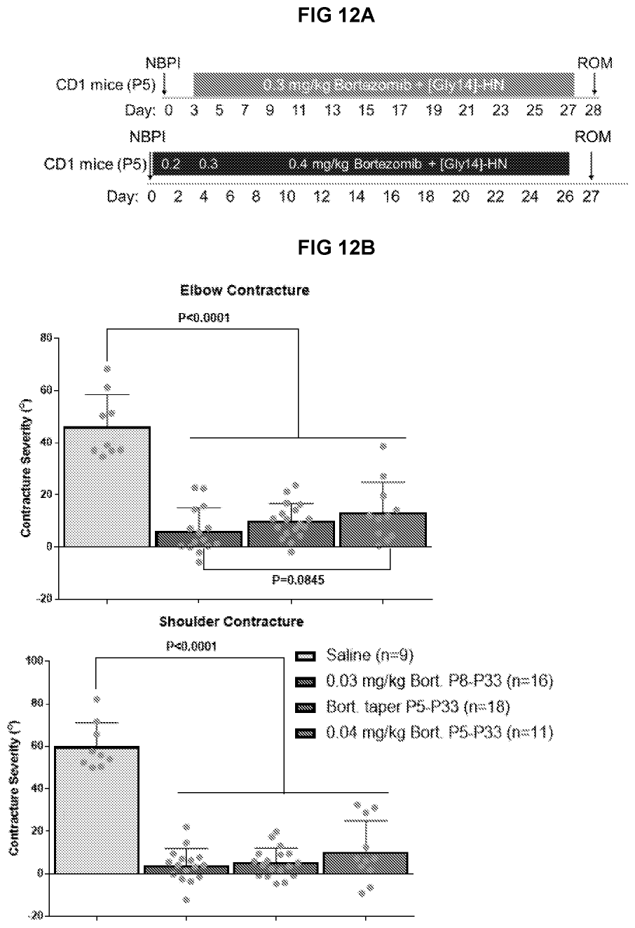

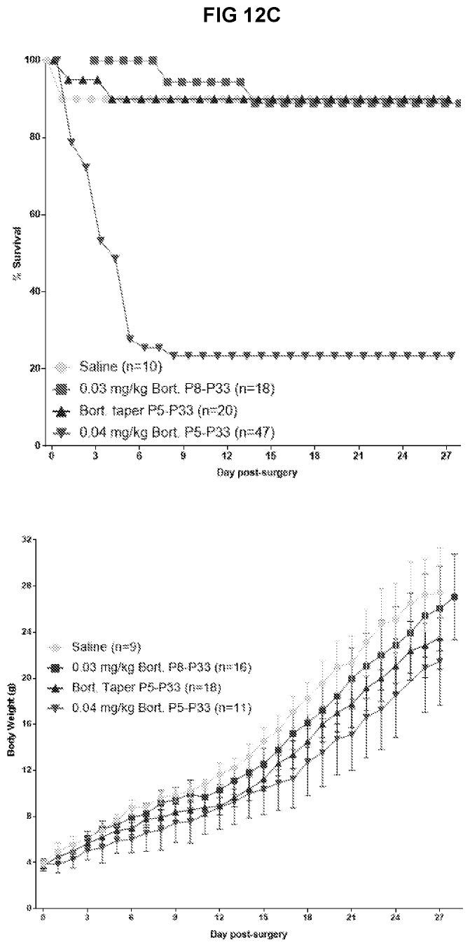

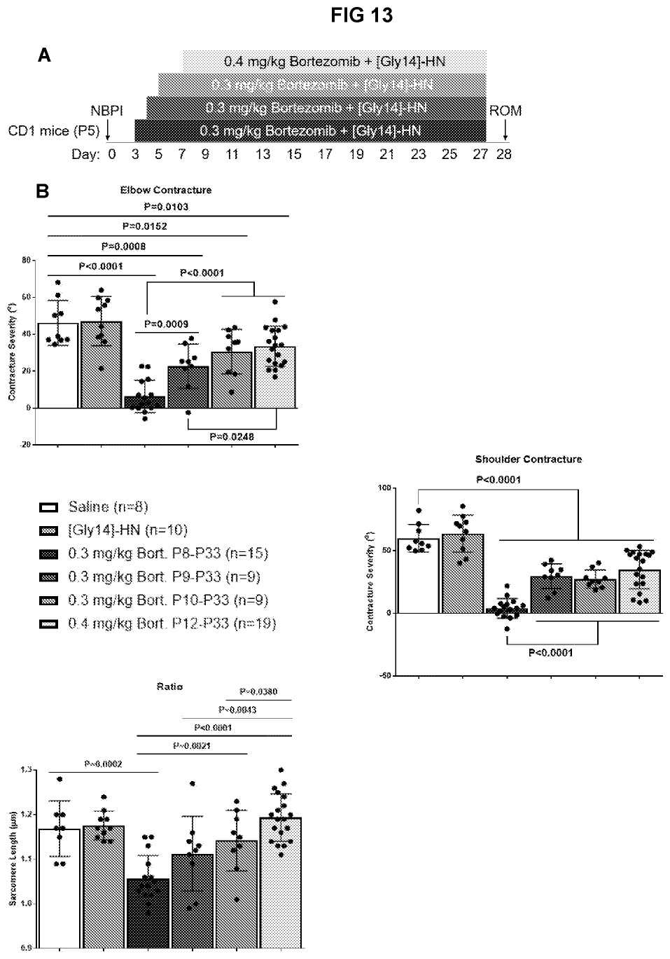

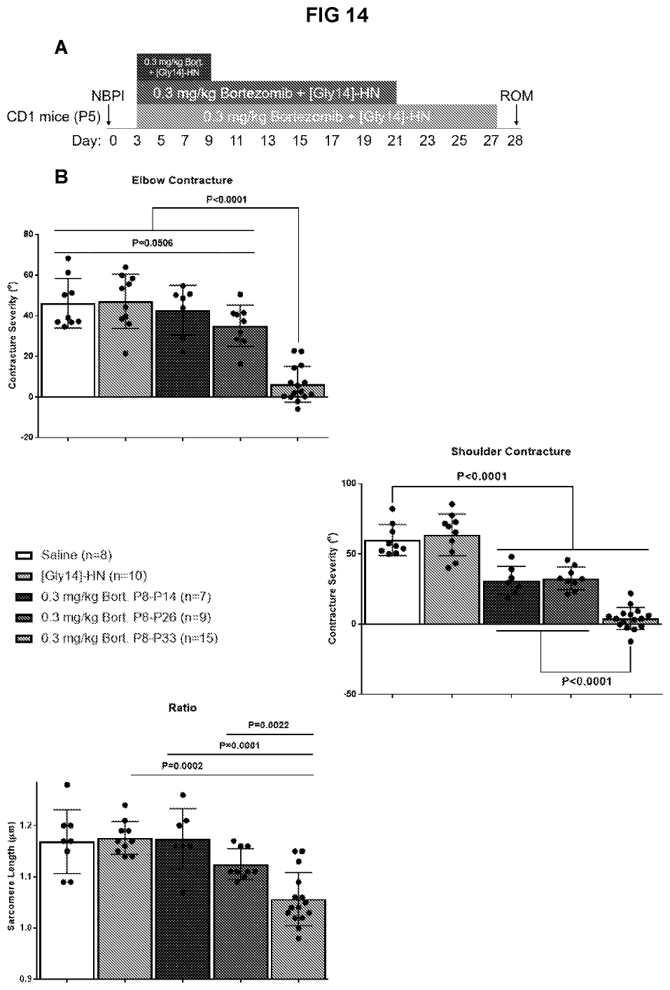

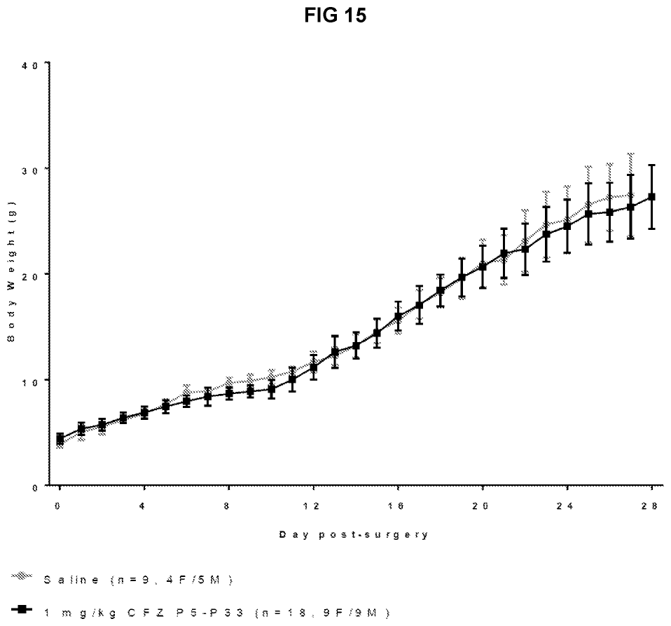

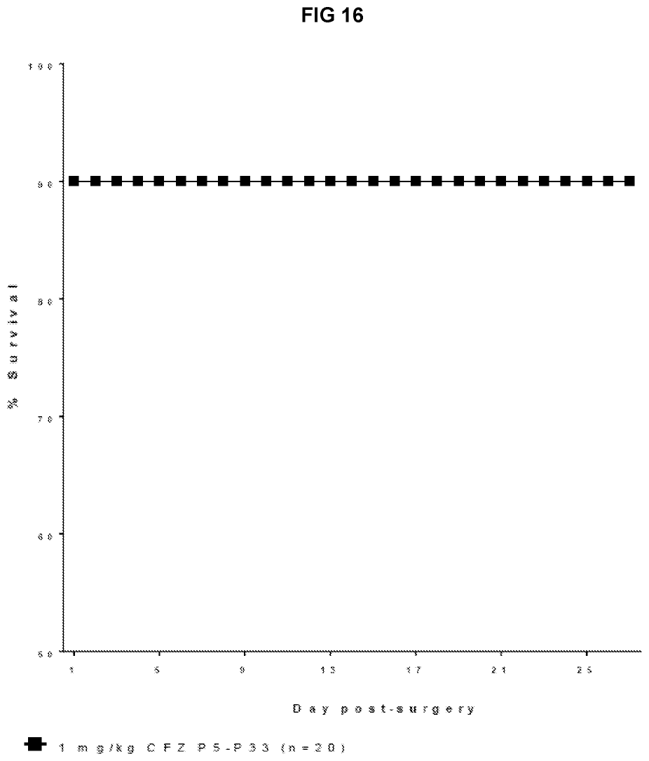

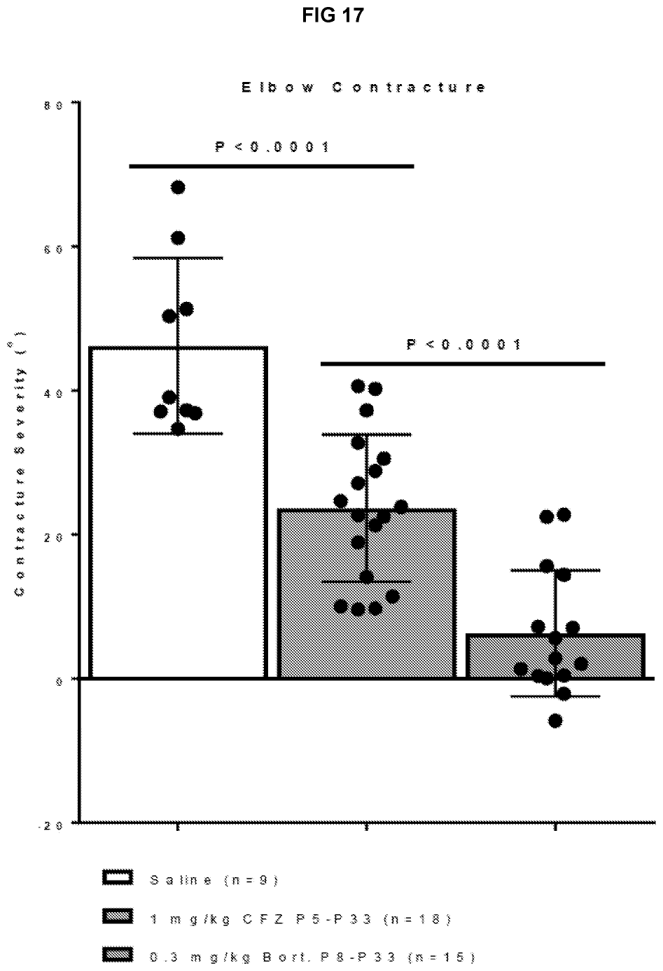

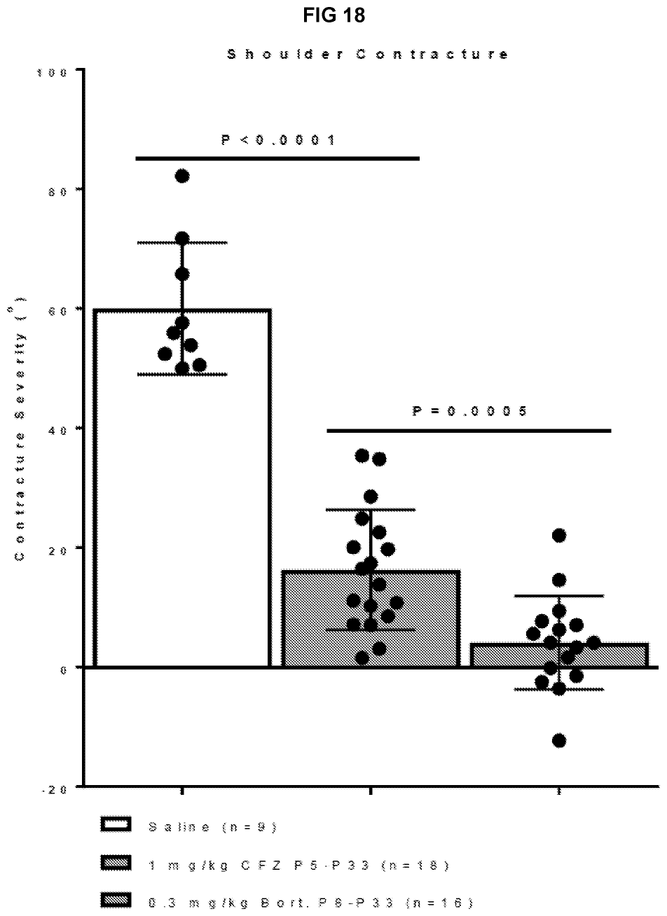

Applicant therefore tested if pharmacologic inhibition of proteasome-mediated protein degradation after NBPI could preserve muscle length and prevent contractures. Following NBPI at P5, the 20S proteasome inhibitor, Bortezomib⁴⁶, was administered at a dose of 0.4 mg/kg body weight every other day from P5 to P33 FIG. 4, A). Bortezomib was co-administered with [Gly¹⁴]-Humanin to mitigate known toxic effects of Bortezomib⁴⁷. Saline and [Gly¹⁴]-Humanin were administered in separate animals as controls. Blinded assessment of shoulder and elbow range of motion in mice 4 weeks after NBPI indicated that Bortezomib rescued the elbow and shoulder contracture phenotypes (FIG. 4, B), significantly reducing shoulder and elbow contracture severity (difference between NBPI and contralateral forelimb passive external rotation and elbow extension, respectively) (FIG. 4, C). [Gly¹⁴]-Humanin had no effect alone. However, Bortezomib treatment caused mortality, mostly in the first week of treatment (FIG. 7, A). To overcome this toxicity, Applicant optimized the dose and timing of Bortezomib. Specifically, Applicant treated WT mice with Bortezomib using the following regimens: 0.2 mg/kg from P5 to P33, 0.3 mg/kg from P5 to P33, 0.4 mg/kg from P8 to P33, and 0.4 mg/kg from P12-P33 (FIG. 7, B). Lowering the dose to 0.2 mg/kg or delaying treatment until P12 eliminated mortality (FIG. 7, C), but while these strategies resulted in less severe contractures compared to saline they were not as efficacious as 0.4 mg/kg Bortezomib administered beginning at P5 (FIG. 7, D). Conversely, lowering the dose to 0.3 mg/kg or initiating treatment at P8 maintained efficacy and partially improved mortality compared to 0.4 mg/kg Bortezomib administered at P5 (FIG. 7, C and D).

Using the above Bortezomib data, Applicant optimized a dosing strategy to maximize efficacy and limit mortality. Applicant treated WT mice with 0.3 mg/kg Bortezomib from P8 to P33 (FIG. 4, D). With this treatment strategy, Applicant observed minimal early death (FIG. 7, E) and optimal efficacy in prevention of contractures (FIG. 4e,f). This therapeutic effect of Bortezomib was accompanied by a rescue of brachialis length, as evidenced by a 70% reduction in the sarcomere elongation caused by NBPI (FIG. 4, G), further indicating that neonatal contractures are caused by impaired longitudinal muscle growth. The findings presented here therefore show that Bortezomib preserves length of denervated muscle and prevents contractures in a dose-dependent manner following NBPI, representing the first ever strategy to prevent neuromuscular contractures by correcting the underlying pathology.

### Discussion

For decades, neuromuscular contractures have been considered a mechanical problem absent any biological explanation, and only palliative mechanical solutions for them have been available. In this work, Applicant demonstrated that the fundamental mechanism leading to contracture development is improper longitudinal muscle growth due to increased proteasome activity. Surprisingly, MuSCs and myonuclear accretion do not control muscle length or contribute to contracture pathology. Remarkably, proteasome inhibition during neonatal growth prevents contractures, representing a paradigm-shifting approach to this debilitating and previously unsolved clinical problem.

The role of myonuclear accretion in adult muscle homeostasis has been explored in recent years, with evidence from MuSC ablation studies suggesting that myonuclear accretion is necessary for normal muscle hypertrophy during overload40,48 and regeneration following injury⁴⁹. However, the role for MuSC-mediated myonuclear accretion in neonatal muscle growth has only been observationally characterized, as ablation of MuSCs in neonatal animals has been complicated by lethality³¹. Nonetheless, myonuclear accretion occurs uniquely during neonatal muscle growth³⁰, during the time frame of contracture development post-NBPI. In addition, myonuclear domain as a function of length remains constant during neonatal growth³⁰, suggesting a tight coupling of myonuclear accretion and sarcomerogenesis. Because of these findings, Applicant initially hypothesized that impaired myonuclear accretion would underlie contracture pathogenesis. Applicant was surprised to find that reduction of myonuclear number through genetic deletion of Myomaker in progenitors does not impair longitudinal muscle growth or cause contractures. Moreover, Applicant found that myonuclear domain as a function of length, measured in serial sarcomeres, is able to increase substantially in the absence of normal myonuclear numbers. These data indicate that dysregulation of the final function of MuSCs, to fuse and contribute a new nucleus to the myofiber, cannot be a major mechanism for impaired longitudinal growth and contracture pathogenesis. However, Applicant did observe dysregulation of MuSCs in terms of increased numbers and proliferative ability potentially suggesting they may respond to or indirectly impact pathogenesis, perhaps through crosstalk with other progenitor populations in muscle⁵⁰.

On the surface, the results suggest the pathways that control longitudinal muscle growth in the neonatal period are similar to what leads to atrophy in adult denervated muscle⁵¹. Indeed, Applicant observed increased levels of MuRF1 and elevated proteasome activity. Moreover, Applicant also found elevated protein synthesis in NBPI muscle, consistent with adult denervation-induced atrophy⁵². Given these similarities between neonatal and adult denervation, it is surprising that proteasome inhibition was able to completely prevent the contracture phenotype in Applicant's model, in contrast to only partial and inconsistent rescue of the loss of muscle mass in adult models of denervation-induced atrophy⁵³. One difference between neonatal denervation and adult denervation is that myonuclear accretion is occurring in the former condition, and could explain the possibly compensatory activation of protein synthesis. Another difference is that denervation of adult muscle is mainly characterized by atrophy in width of myofibers, whereas neonatal neuromuscular contractures are due to reduced longitudinal growth. Indeed, the data indicate that contracture prevention is accompanied by nearly complete rescue of muscle length. Thus, the path to an effective treatment for neonatal neuromuscular contractures following NBPI may be more straightforward than mitigating adult muscle atrophy, as longitudinal growth may be more tightly (or more likely uniquely occurring in the neonatal period) controlled by protein degradation.

While bortezomib is currently in use for adult cancer treatment and is in clinical trials in children⁴⁶, it is associated with toxicity. Applicant minimized toxicity by adjusting the dose and timing of treatment and by co-administering [Gly¹⁴]-Humanie. By defining the necessary treatment window for preventing contractures, cumulative toxicity from long-term administration may be limited. Indeed, denervation outside the neonatal period does not cause contractures¹⁹, suggesting that a finite window of bortezomib treatment may be sufficient. Furthermore, newer generation proteasome inhibitors have been developed, with more favorable toxicity profiles⁵⁴. Finally, exploring the complex regulatory network governing protein dynamics may yield additional targets to restore anabolic proteostasis in neonatally denervated muscle. Nonetheless, Applicant's findings provide proof of concept that proteasome inhibition is sufficient to prevent contractures following NBPI.

The findings of this study also provide a foundation to develop strategies for preventing contractures in other neuromuscular disorders. Contractures in cerebral palsy are similarly characterized by impaired longitudinal muscle growth, indicated by sarcomere elongation identical to that seen in Applicant's model following NBPI. Although the neurologic pathology differs between NBPI and CP, the perinatal age of onset is similar. Similarly, muscle contractures occur following other early childhood neuromuscular disorders, such as spinal muscular atrophy⁵⁵, especially the types with perinatal onset. Therefore, although the relationships between innervation and proteostasis in the neonatal period are not fully elucidated in NBPI or CP, future studies confirming the efficacy of proteasome inhibition in animal models and clinical pediatric populations could ultimately render obsolete the destructive surgeries currently required to alleviate a wide variety of disabling neuromuscular contractures and the secondary skeletal deformities that result from them.

### Methods

### NBPI Surgical Model

All animal procedures were approved by Cincinnati Children's Hospital Medical Center's Institutional Animal Care and Use Committee. Unilateral global (C5-T1) NBPIs were created by surgical extraforaminal nerve root excision in 5-day-old CD-1 mice (Charles River) under general anesthesia. Deficits in motor function were validated post-operatively and again prior to sacrifice to ensure only animals with permanent motor deficits were included for analysis. Elbow and shoulder (where indicated) range of motion were measured immediately post-sacrifice using a validated digital photography technique in order to confirm the presence of elbow flexion and shoulder internal rotation contractures¹⁶. Mice were euthanized by CO₂ asphyxiation, except at postnatal day 5 and 12 time points, where isoflurane overdose was utilized.

### Immunohistochemistry

Bilateral biceps muscles were harvested, fixed in 10% neutral buffered formalin (NBF) for 1 hour, then cryoprotected in sucrose prior to snap freezing in optimum cutting temperature (OCT). Frozen sections (10 μm) were taken from the mid-muscle belly region and treated with 10 mM sodium citrate, pH 6.0 heat-mediated antigen retrieval in a rice steamer for 5 min. Slides were permeabilized in 0.4% Triton X-100/PBS for 10 minutes and blocked in 10% normal donkey serum (NDS; Jackson ImmunoResearch) and 1% bovine serum albumin (BSA), then blocked in donkey anti-mouse IgG Fab fragment (1:50, Jackson ImmunoResearch) (with 1% NDS and 1% BSA) in PBS for 2 hours each. Primary antibodies were mouse anti-Pax7 (1:100, sc-81648, Santa Cruz Biotechnology) and rabbit anti-MyoD (1:50, sc-760, Santa Cruz Biotechnology), in PBS containing 1% NDS and 1% BSA and incubated overnight at 4° C. Secondary antibodies were donkey anti-mouse IgG-DyLight 549 (1:800, 715-505-150, Jackson ImmunoResearch) and donkey anti-rabbit IgG-DyLight 649 (1:800, 711-495-152, Jackson ImmunoResearch), diluted in PBS containing 1% NDS, 1% BSA and 1 μg/mL 4′,6-diamidino-2-phenylindole dihydrochloride (DAPI; Sigma-Aldrich) and incubated for at least 1 hour. Slides were mounted in Vectashield antifade mounting medium (Vector Laboratories) and imaged by widefield epifluorescence on an Axioplan 2 imaging microscope with the Plan Apochromat 20× objective using AxioVision software (Carl Zeiss Microscopy). Three images per muscle sample from 4 mice were analyzed using Imaris software (Bitplane).

CD-1 mice were given 5-bromo-2′-deoxyuridine (BrdU; 00-0103, Invitrogen) by daily intraperitoneal (IP) injections (10 μL/g body weight) starting from post-NBPI day 1. At 2 weeks post-NBPI (24 h following the last BrdU injection), bilateral biceps muscles were harvested and snap frozen in OCT. Frozen sections (10 μm) were taken from the mid-muscle belly region, fixed in 4% paraformaldehyde (PFA) in PBS for 5 minutes and treated with 2N HCl, pH 0.6-0.9 for 10 minutes, permeabilized in 0.5% Triton X-100/PBS for 6 minutes, and blocked as described above. Primary antibodies were mouse anti-Pax7, rat anti-BrdU (1:200, ab6326, Abcam) and rabbit anti-Dystrophin (1:250, ab15277, Abcam), diluted in PBS containing 1% NDS and 1% BSA and incubated overnight at 4° C. Secondary antibodies were donkey anti-mouse IgG-Alexa Fluor 555 (1:800, A-31570, Invitrogen), donkey anti-rat-Alexa Fluor 488 (1:800, 712-545-153, Jackson ImmunoResearch) and donkey anti-rabbit-Alexa Fluor 647 (1:800, 711-605-152, Jackson ImmunoResearch), diluted in PBS containing 1% NDS, 1% BSA and 1 μg/mL DAPI, and incubated for at least 1 h. Slides were mounted in Prolong Gold antifade mountant (Life Technologies) and imaged on a Nikon Eclipse Ti inverted microscope with the Plan Apo VC 20× DIC N2 objective on a Nikon MR confocal using the 405 nm, 488 nm, 561 nm, and 638 nm lasers and NIS-Elements imaging software (Nikon Instruments). Three images (˜100 muscle fibers) per muscle sample from 7 mice were analyzed using the Fiji program⁵⁶ (https://fiji.sc/; Cell Counter plug-in).

### Genetically Modified Mice

NBPIs were created as described above in 5-day-old Pax7^{CreER}; Rosa26^{LacZ} (double homozygous) transgenic mice (stock numbers 017763 and 009427, The Jackson Laboratory)^{57,58}. Beta-galactosidase reporter gene expression was induced in Pax7⁺ with a single dose of tamoxifen (0.5 mg/g body weight in corn oil; T5648, Sigma-Aldrich) administered by oral gavage 2 days post-NBPI (P7). Bilateral biceps muscles were harvested at 2 weeks post-NBPI, snap frozen in OCT and 10 μm frozen sections were taken from the muscle belly region proximal to the shoulder. Sections were then fixed in 2% PFA/PBS for 5 minutes before using a standard 5-bromo-4-chloro-3-indolyl-β-D-galactoside (X-Gal) staining protocol with overnight colorimetric development. Slides were mounted in Prolong Gold antifade mountant and imaged on a Nikon 90i microscope with the Plan Apo 20× DIC M objective, Photometrics CoolSNAP HQ2 monochromatic camera and NIS-Elements imaging software. Color RGB images were generated by setting exposures of the TRITC, GFP and DAPI filters (with epifluorescence shutters closed) to generate a white background image when merged (manual white-color balance). The colored RGB images were merged and three images (˜100 muscle fibers) per muscle sample from 7 mice were analyzed using the Fiji program (Cell Counter plug-in).

Mymk^{scKO} mice were generated by crossing Mymk^{loxP/loxP} mice and Pax7^{CreER} mice in the to yield Mymkl^{oxP/loxP} ; Pax7^{CreERT2} mice^{40,41,59.} These genetically modified alleles are in the C57B16 background. Mymk^{loxP/loxP} mice served as controls. To delete Mymk in MuSCs, mice were administered 200 mg tamoxifen (10 mg/ml in 90% corn oil/10% EtOH) by IP injection at P0. Muscle was harvested at P5 for expression analysis to confirm down-regulation of Mymk. RNA was isolated from the gastrocnemius muscle using Trizol (Invitrogen), and cDNA was synthesized using MultiScribe reverse transcriptase with random hexamer primers (Applied Biosystems). Gene expression was assessed using PowerUp SYBR Green Master Mix (Applied Biosystems), and performed on a 7900HT fast real-time PCR machine (Applied Biosystems). qPCR was performed using the following primers for Mymk: forward, 5′-ATCGCTACCAAGAGGCGTT-3′ (SEQ ID NO: 1); reverse, 5′-CACAGCACAGACAAACCAGG-3′ (SEQ ID NO: 2). Results were normalized to glyceraldehyde phosphate dehydrogenase (GAPDH) using the following primers: forward, 5′-TGCGACTTCAACAGCAACTC-3′ (SEQ ID NO: 3); reverse, 5′-GCCTCTCTTGCTCAGTGTCC-3′ (SEQ ID NO: 4).

To isolate single myofibers, extensor digitorum longus (EDL) and biceps muscles were harvested and incubated in high-glucose DMEM (Hyclone Laboratories) containing 0.2% collagenase Type I (Sigma-Aldrich) at 37° C. for 45-60 minutes. After 40 minutes of incubation, muscles were gently triturated to loosen the digesting myofibers, and then returned to the incubator for up to 60 total minutes. After incubation, muscles were removed from the 0.2% collagenase/DMEM solution and placed into PBS. To isolate single myofibers, muscles were triturated using pipettes with bores of decreasing sizes until myofibers shed from the muscle. Single myofibers were collected and fixed in 4% PFA/PBS for 20-30 minutes at room temperature, and subsequently stored in PBS at 4° C. To analyze the number of myonuclei, myofibers were permeabilized in 0.2% Triton X-100/PBS for 10 minutes at room temperature, washed three times in PBS, and mounted on slides with VectaShield containing DAPI (Vector Laboratories). Myofibers were imaged using a Nikon SpectraX widefield microscope with the 10× objective. Myonuclei were counted in 3D reconstructed images using Imaris software (Bitplane). 15-20 myofibers were analyzed per mouse in each muscle.

Sarcomere lengths were measured from single muscle fibers, acquiring 6 images per fiber by differential interference contrast (DIC) microscopy on a Nikon Eclipse Ti-E inverted microscope with the Plan Apo λ40× objective (Nikon Instruments), Xyla 4.2 megapixel, 16-bit sCMOS monochromatic camera (Andor/Oxford Instruments) and NIS-Elements imaging software (Nikon Instruments). A series of 10 sarcomeres were measured per image in AxioVision software and an average sarcomere length was then determined for each fiber.

Mouse limbs harvested 4 weeks post-NBPI were processed on cork at 90° elbow flexion (confirmed by digital x-ray) prior to fixation in 10% NBF as described previously¹⁶. Brachialis muscles were then removed, digested in 15% sulfuric acid for 30 minutes to obtain muscle bundles¹⁶, and imaged for sarcomere length measurement by DIC microscopy as described above.

### Gene Expression Analysis of NBPI Muscle

Total RNA was extracted from snap frozen bilateral biceps muscles from 3 mice harvested 3 weeks post-NBPI using the ReliaPrep RNA tissue miniprep system (Promega). The concentration and quality of the RNA samples were determined using the Bioanalyzer (Agilent), and 10 ng of each amplified using the Ovation RNA-Seq system V2 (NuGEN), constructed into cDNA libraries using the Nextera XT DNA sample preparation kit (Illumina), and sequenced on the HiSeq 2500 system (Illumina, Paired-End 75 bp Flow Cell) to a depth of at least 35-40 million reads. Resulting FASTQ sequences were pseudoaligned against the Mus musculus transcriptome (EnsMart72/mm 10) using the Kallisto⁶⁰ program and analyzed with the AltAnalyze⁶¹ program (transcripts per million (TPM) filtered by adjP value and 2-fold change in gene expression). Gene ontology analysis was performed on the genes that changed by log₂ fold change to select for genes that exhibited the most robust differential regulation. Here, 336 genes were up-regulated and 21 genes were down-regulated. The 336 up-regulated genes were analyzed for enrichment of biological processes using the Gene Ontology Consortium (http://www.geneontology.org)^{62,63}.

To assess MuRF1 transcript levels, RNA was extracted from snap frozen bilateral biceps muscles from 6 mice harvested 2 weeks post-NBPI as described above, and 500 ng of each was used in first strand cDNA synthesis using the GoScript reverse transcription system (Promega) with both oligo(dT)15 and random primers (0.5 μg each primer/reaction) carried out for 1 h at 50° C., followed by heat inactivation. Primers for PCR were designed using the Primer3^{64,65} program (http://bioinfo.ut.ee/primer3/) so that one primer per set bound across an exon-exon boundary. MuRF1 (Trim63) gene target transcript Trim63-202 (ENSMUST00000105875.7) forward: 5′-GGAGAACCTGGAGAAGCAGC-3′ (SEQ ID NO: 5) and reverse: 5′-TAGGGATTCGCAGCCTGGAA-3′ (SEQ ID NO: 6); and Atp5j gene normalizer transcript Atp5j-201 (ENSMUST00000023608.13) forward: 5′-TCAGTGCAAGTACAGAGACTCA-3′ (SEQ ID NO: 7) and reverse: 5′-GCCTGTCGCTTTGATTTGTACT-3′ (SEQ ID NO: 8). The gene Atp5j (ENSMUSG00000022890) was chosen for normalization due to finding that it was expressed at similar high levels between 3 week post-NBPI and contralateral control biceps muscles in the RNA-Sequencing data.

Each 20 μl PCR contained: 1× GoTaq qPCR master mix (Promega; containing a proprietary dye detected with the SYBR channel), 0.2 μl CXR reference dye (detected with the ROX channel), 5 pmol each primer and 2 μl cDNA (diluted 1:10); and was carried out in a 96-well plate on the StepOnePlus real-time PCR system (Applied Biosystems). PCR cycling was: hot-start activation at 95° C. for 2 minutes, 40 cycles of denaturation at 95° C. for 15 seconds, annealing at 54° C. (Atp5j) or 56° C. (Trim63) for 15 seconds and extension at 60° C. for 1 minute (data acquisition at the end of step to measure rate of amplification); final dissociation for 1 cycle at 95° C. for 15 seconds, and then Melt Curve analysis starting at 60° C. for 1 minute then +0.3° C. for 15 seconds per temperature interval until 95° C. with continuous data acquisition to confirm the generation of a single PCR product. Average Cₜ was determined from triplicate reactions using the StepOne software (Applied Biosystems) and fold difference in gene expression determined using the Comparative Cₜ (ΔΔCₜ) method, with correction of PCR efficiency (E=10_{[-1/slope]}) between target (Trim63) and normalizer (Atp5j) primer sets determined from a 4-point standard curve (1:5, 1:50, 1:500 and 1:5000 dilutions of pooled cDNA from test bilateral biceps muscles from one mouse harvested at week 3 post-NBPI prepared as described above) included in each primer set reaction run, using the following equations⁶⁶: ΔC_{t target}=C_{t GOI}_{^{c}}−C_{t GOI}_{^{s}}, ΔC_{t normalizer}=C_{t norm}_{^{c}}−C_{t norm}^{s}, and fold difference=(E_{target})^{ΔCt target}/(E_{normalizer})^{ΔCt normalizer}, where s represents individual mouse samples from bilateral biceps at 2 weeks post-NBPI (6 mice), and c represents the calibrator sample derived from unilateral biceps from unoperated mice that are age-matched to 3 weeks post-NBPI (average of 3 mice).

### Analysis of Protein Dynamics Post-NBPI

NBPIs were created as described above in 5-day-old mice. Surface sensing of translation (SUnSET)^{67,68} was performed by administration of puromycin (21.8 mg/kg body weight; P7255, Sigma-Aldrich) by IP injection 30 minutes prior to sacrifice at weekly time points, beginning immediately post-operatively until 4 weeks post-NBPI. Total proteins were extracted from snap frozen bilateral biceps muscles using radioimmunoprecipitation assay (RIPA) buffer containing cOmplete ULTRA proteasome inhibitor cocktail (Roche) and PhosSTOP phosphatase inhibitor cocktail (Roche) and centrifuged at 20000×g for 20 minutes at 4° C. Proteins were then precipitated with acetone and resuspended in 1× Laemmli sample buffer (161-0737, Bio-Rad) prepared with 2-mercaptoethanol and RIPA buffer, and heat denatured for 5 minutes at 95° C. Equal protein loads were run on 4-15% Mini-PROTEAN TGX Gels (456-1086, Bio-Rad) in 25 mM Tris, 192 mM glycine, 0.1% SDS running buffer, and transferred to Immobilon-FL polyvinylidene fluoride (PVDF; IPFL10100, Millipore) in 25 mM Tris, 192 mM glycine, 20% methanol transfer buffer. Western blot analysis was carried out using the following antibodies: rat anti-Puromycin (1:1000, MABE341, Sigma-Aldrich), rabbit anti-K48-linkage specific polyubiquitin (1:1000, 80815, Cell Signaling), rabbit anti-Skeletal Muscle Actin (1:1000, ab15263, Abcam), mouse anti-Fast Myosin (1:1000, ab51263, Abcam) and mouse anti-Slow Myosin (1:5000, ab11083, Abcam). Western blots were detected using species-specific secondary antibodies raised in donkey and conjugated to either Alexa Fluor 680 or 790 (1:100000, Jackson ImmunoResearch) imaging with the Odyssey CLx, and signal intensities measured using the Image Studio Lite program (LI-COR Biosciences). Western blot signals were normalized to gel protein load.

To assay proteasome activity, bilateral biceps muscles from 6 mice harvested 2 weeks post-NBPI were snap frozen, extracted in 20 mM Tris-HCl, pH 7.2, 0.1 mM EDTA, 1 mM 2-mercaptoethanol, 5 mM ATP, 20% glycerol, 0.04% Nonidet P-40 and centrifuged at 13000×g for 15 minutes at 4° C.⁶⁹. Protein concentration was determined using the Pierce 660 nm protein assay kit (Thermo Scientific) and 25 μg total protein per muscle used to assay the chymotrypsin-like activity of the 20S proteasome beta-5 catalytic subunit through detection of 7-Amino-4-methylcoumarin (AMC) fluorescence by cleavage of the peptide substrate Suc-LLVY-AMC (S-280, Boston Biochem) in 25 mM HEPES, pH 7.5, 0.5 mM EDTA, 0.05% NP-40, 0.001% SDS. Assay design was based on the Chemicon kit (APT280) and duplicate reactions were carried out in a white opaque polystyrene 96-well plate for 2 hours at 37° C., with endpoint fluorescence measured at 380/460 nm in a SpectraMax M5 microplate reader (Molecular Devices). Relative fluorescence units (RFU) were then calculated per μg protein.

### Bortezomib Treatment

Mice were treated either with saline (as the vehicle; 0.9% Sodium Chloride Injection USP, Hospira), [Gly¹⁴]-Humanin G ([Gly14]-HN; 1 μg/dose; H6161, Sigma-Aldrich) alone or co-administered with Bortezomib (0.2-0.4 mg/kg body weight; 5043140001, Sigma-Aldrich) by IP injection starting immediately post-operative or delayed by 3/7 days (P8/12 start), and injected every other day with sacrifice at 4 weeks post-NBPI (24 h following the last IP injection). The use of littermate controls was rejected due to the risk of treatment cross-contamination either through direct contact or by ingestion from their mother, and [Gly14]-HN was included to mitigate the toxicity that has been reported for Bortezomib⁴⁷. Deficits in motor function were confirmed as described above, and measurement of shoulder and elbow range of motion was measured immediately post-sacrifice¹⁶ with blinding to the treatment group. Mouse limbs harvested 4 weeks post-NBPI were positioned on cork for processing of bilateral brachialis muscles for DIC microscopy as described above for measurement of muscle sarcomere length.

### Statistics

For all continuous data, outliers were detected a priori by Grubbs' test and excluded. All continuous data with n>3 animals were tested for normality with the Shapiro-Wilk test. Normally distributed data and data with n=3 were compared with two-tailed Student's t-test, paired where parameters were compared between forelimbs (NBPI versus contralateral) in individual animals, and unpaired when parameters were compared between animals. Non-normally distributed data were compared using Mann-Whitney U tests for unpaired data or Wilcoxon signed rank tests for paired analyses where parameters were compared between forelimbs (NBPI versus contralateral). All data are presented as mean±s.d. The degree of significance between data sets is depicted as follows: *P<0.05, **P<0.01, ***P<0.001, ****P<0.0001. A priori power analyses based on prior work were performed for the phenotypic variables of contracture severity, determining that 6 mice per group were required for at least 80% power to detect a 10° difference in contractures and a 0.2 μM difference in sarcomere lengths between experimental conditions.

### REFERENCES

1. Cornwall, R. & Waters, P. M. Pediatric Brachial Plexus Palsy. in Green's Operative Hand Surgery, Vol. 2 (eds. Wolfe, S. W., Hotchkiss, R. N., Pederson, W. C., Kozin, S. K. & Cohen, M. S.) 1391-1424 (Elsevier, Philadelphia, 2017).

2. Yeargin-Allsopp, M., et al. Prevalence of cerebral palsy in 8-year-old children in three areas of the United States in 2002: a multisite collaboration. Pediatr 121, 547-554 (2008).

3. Foad, S. L., Mehlman, C. T. & Ying, J. The epidemiology of neonatal brachial plexus palsy in the United States. J Bone Joint Surg Am 90, 1258-1264 (2008).

4. Graham, H. K., et al. Cerebral palsy. Nature reviews. Disease primers 2, 15082 (2016).

5. Waters, P. M., Smith, G. R. & Jaramillo, D. Glenohumeral deformity secondary to brachial plexus birth palsy. J Bone Joint Surg Am 80, 668-677 (1998).

6. Sibinski, M., Wozniakowski, B., Drobniewski, M. & Synder, M. Secondary gleno-humeral joint dysplasia in children with persistent obstetric brachial plexus palsy. Int Orthop 34, 863-867 (2010).

7. Kozin, S. H. Correlation between external rotation of the glenohumeral joint and deformity after brachial plexus birth palsy. J Pediatr Orthop 24, 189-193 (2004).

8. Larnert, P., Risto, O., Hagglund, G. & Wagner, P. Hip displacement in relation to age and gross motor function in children with cerebral palsy. J Child Orthop 8, 129-134 (2014).

9. Miller, F., Cardoso Dias, R., Dabney, K. W., Lipton, G. E. & Triana, M. Soft-tissue release for spastic hip subluxation in cerebral palsy. J Pediatr Orthop 17, 571-584 (1997).

10. Waters, P. M. Update on management of pediatric brachial plexus palsy. J Pediatr Orthop 25, 116-126 (2005).

11. Janice, M. Q. & Alwyn, A. Musculoskeletal problems in cerebral palsy. Curr Paediatr 15, 9-14 (2005).

12. Newman, C. J., Morrison, L., Lynch, B. & Hynes, D. Outcome of subscapularis muscle release for shoulder contracture secondary to brachial plexus palsy at birth. J Pediatr Orthop 26, 647-651 (2006).

13. Tilton, A. H. Therapeutic interventions for tone abnormalities in cerebral palsy. NeuroRx 3, 217-224 (2006).

14. van der Sluijs, J. A., et al. Treatment of internal rotation contracture of the shoulder in obstetric brachial plexus lesions by subscapular tendon lengthening and open reduction: early results and complications. J Pediatr Orthop B 13, 218-224 (2004).

15. Orendurff, M. S., Aiona, M. D., Dorociak, R. D. & Pierce, R. A. Length and force of the gastrocnemius and soleus during gait following tendo Achilles lengthenings in children with equinus. Gait Posture 15, 130-135 (2002).

16. Nikolaou, S., Peterson, E., Kim, A., Wylie, C. & Cornwall, R. Impaired growth of denervated muscle contributes to contracture formation following neonatal brachial plexus injury. J Bone Joint Surg Am 93, 461-470 (2011).

17. Nikolaou, S., Hu, L. & Cornwall, R. Afferent Innervation, Muscle Spindles, and Contractures Following Neonatal Brachial Plexus Injury in a Mouse Model. J Hand Surg Am 40, 2007-2016 (2015).

18. Nikolaou, S., et al. Contribution of denervated muscle to contractures following neonatal brachial plexus injury: Not just muscle fibrosis. Muscle Nerve 49, 398-404 (2014).

19. Weekley, H., et al. The effects of denervation, reinnervation, and muscle imbalance on functional muscle length and elbow flexion contracture following neonatal brachial plexus injury. J Orthop Res 30, 1335-1342 (2012).

20. Crouch, D. L., et al. Biomechanical Basis of Shoulder Osseous Deformity and Contracture in a Rat Model of Brachial Plexus Birth Palsy. J Bone Joint Surg Am 97, 1264-1271 (2015).

21. Mascarenhas, V. V., et al. The role of subscapularis muscle denervation in the pathogenesis of shoulder internal rotation contracture after neonatal brachial plexus palsy: A study in a rat model. J Orthop Res 32, 1675-1679 (2014).

22. Eismann, E. A., Little, K. J., Laor, T. & Cornwall, R. Glenohumeral abduction contracture in children with unresolved neonatal brachial plexus palsy. J Bone Joint Surg Am 97, 112-118 (2015).

23. Russo, S. A., et al. Scapulothoracic and glenohumeral contributions to motion in children with brachial plexus birth palsy. J Shoulder Elbow Surg 23, 327-338 (2014).

24. Russo, S. A., et al. Limited glenohumeral cross-body adduction in children with brachial plexus birth palsy: a contributor to scapular winging. J Pediatr Orthop 35, 240-245 (2015).

25. Cheng, W., Cornwall, R., Crouch, D. L., Li, Z. & Saul, K. R. Contributions of muscle imbalance and impaired growth to postural and osseous shoulder deformity following brachial plexus birth palsy: a computational simulation analysis. J Hand Surg Am 40, 1170-1176 (2015).

26. Crouch, D. L., Plate, J. F., Li, Z. & Saul, K. R. Computational sensitivity analysis to identify muscles that can mechanically contribute to shoulder deformity following brachial plexus birth palsy. J Hand Surg Am 39, 303-311 (2014).

27. Friden, J. & Lieber, R. L. Spastic muscle cells are shorter and stiffer than normal cells. Muscle Nerve 27, 157-164 (2003).

28. Ho, E. S. Evaluation of pediatric upper extremity peripheral nerve injuries. J Hand Ther 28, 135-142 (2015).

29. Dumont, N. A., Bentzinger, C. F., Sincennes, M. C. & Rudnicki, M. A. Satellite Cells and Skeletal Muscle Regeneration. Comprehensive Physiology 5, 1027-1059 (2015).

30. White, R. B., Bierinx, A. S., Gnocchi, V. F. & Zammit, P. S. Dynamics of muscle fibre growth during postnatal mouse development. BMC Dev Biol 10, 21 (2010).

31. Pallafacchina, G., Blaauw, B. & Schiaffino, S. Role of satellite cells in muscle growth and maintenance of muscle mass. Nutr Metab Cardiovasc Dis 23, S12-18 (2012).

32. de Castro Rodrigues, A. & Schmalbruch, H. Satellite cells and myonuclei in long-term denervated rat muscles. Anat Rec 243, 430-437 (1995).

33. Dayanidhi, S., et al. Reduced satellite cell number in situ in muscular contractures from children with cerebral palsy. J Orthop Res 33, 1039-1045 (2015).

34. Kinney, M. C., et al. Reduced skeletal muscle satellite cell number alters muscle morphology after chronic stretch but allows limited serial sarcomere addition. Muscle Nerve 55, 384-392 (2017).

35. Mathewson, M. A. & Lieber, R. L. Pathophysiology of muscle contractures in cerebral palsy. Phys Med Rehabil Clin N Am 26, 57-67 (2015).

36. Smith, L. R., Chambers, H. G. & Lieber, R. L. Reduced satellite cell population may lead to contractures in children with cerebral palsy. Dev Med Child Neurol 55, 264-270 (2013).

37. de Castro Rodrigues, A., Geuna, S., de Mattos Rodrigues, S. P., Silva, M. D. & Aragon, F. F. Satellite cells and myonuclei in neonatally denervated rat muscle. Ital J Anat Embryol 107, 51-56 (2002).

38. Domenighetti, A. A., et al. Loss of myogenic potential and fusion capacity of muscle stem cells isolated from contractured muscle in children with cerebral palsy. Am J Physiol Cell Physiol 315, C247-c257 (2018).

39. Millay, D. P., et al. Myomaker is a membrane activator of myoblast fusion and muscle formation. Nature 499, 301¬305 (2013).

40. Goh, Q. & Millay, D. P. Requirement of myomaker-mediated stem cell fusion for skeletal muscle hypertrophy. eLife 6 (2017).

41. Millay, D. P., Sutherland, L. B., Bassel-Duby, R. & Olson, E. N. Myomaker is essential for muscle regeneration. Genes Dev 28, 1641-1646 (2014).

42. Felder, A., Ward, S. R. & Lieber, R. L. Sarcomere length measurement permits high resolution normalization of muscle fiber length in architectural studies. J Exp Biol 208, 3275-3279 (2005).

43. Furuno, K., Goodman, M. N. & Goldberg, A. L. Role of different proteolytic systems in the degradation of muscle proteins during denervation atrophy. J Biol Chem 265, 8550-8557 (1990).

44. Bodine, S. C. & Baehr, L. M. Skeletal muscle atrophy and the E3 ubiquitin ligases MuRF1 and MAFbx/atrogin-1. Am J Physiol Endocrinol Metab 307, E469-484 (2014).

45. Meng, L., et al. Epoxomicin, a potent and selective proteasome inhibitor, exhibits in vivo antiinflammatory activity. Proc Natl Acad Sci USA 96, 10403-10408 (1999).

46. Citrin, R., Foster, J. B. & Teachey, D. T. The role of proteasome inhibition in the treatment of malignant and non-malignant hematologic disorders. Expert Rev Hematol 9, 873-889 (2016).

47. Cohen, P. New role for the mitochondrial peptide humanin: protective agent against chemotherapy-induced side effects. Journal of the National Cancer Institute 106, dju006 (2014).

48. Egner, I. M., Bruusgaard, J. C. & Gundersen, K. Satellite cell depletion prevents fiber hypertrophy in skeletal muscle. Development 143, 2898-2906 (2016).

49. Relaix, F. & Zammit, P. S. Satellite cells are essential for skeletal muscle regeneration: the cell on the edge returns centre stage. Development 139, 2845-2856 (2012).

50. Fry, C. S., Kirby, T. J., Kosmac, K., McCarthy, J. J. & Peterson, C. A. Myogenic Progenitor Cells Control Extracellular Matrix Production by Fibroblasts during Skeletal Muscle Hypertrophy. Cell Stem Cell 20, 56-69 (2017).

51. Sandri, M. Protein breakdown in muscle wasting: role of autophagy-lysosome and ubiquitin-proteasome. Int J Biochem Cell Biol 45, 2121-2129 (2013).

52. Quy, P. N., Kuma, A., Pierre, P. & Mizushima, N. Proteasome-dependent activation of mammalian target of rapamycin complex 1 (mTORC1) is essential for autophagy suppression and muscle remodeling following denervation. J Biol Chem 288, 1125-1134 (2013).

53. Beehler, B. C., Sleph, P. G., Benmassaoud, L. & Grover, G. J. Reduction of skeletal muscle atrophy by a proteasome inhibitor in a rat model of denervation. Exp Biol Med (Maywood) 231, 335-341 (2006).

54. Yong, K., Gonzalez-McQuire, S., Szabo, Z., Schoen, P. & Hajek, R. The start of a new wave: Developments in proteasome inhibition in multiple myeloma. European journal of haematology (2018).

55. Wang, H. Y., Ju, Y. H., Chen, S. M., Lo, S. K. & Jong, Y. J. Joint range of motion limitations in children and young adults with spinal muscular atrophy. Arch Phys Med Rehabil 85, 1689-1693 (2004).

56. Schindelin, J., et al. Fiji: an open-source platform for biological-image analysis. Nat Methods 9, 676-682 (2012).

57. Murphy, M. M., Lawson, J. A., Mathew, S. J., Hutcheson, D. A. & Kardon, G. Satellite cells, connective tissue fibroblasts and their interactions are crucial for muscle regeneration. Development 138, 3625-3637 (2011).

58. Soriano, P. Generalized lacZ expression with the ROSA26 Cre reporter strain. Nat Genet 21, 70-71 (1999).

59. Lepper, C., Conway, S. J. & Fan, C. M. Adult satellite cells and embryonic muscle progenitors have distinct genetic requirements. Nature (2009).

60. Bray, N. L., Pimentel, H., Melsted, P. & Pachter, L. Near-optimal probabilistic RNA-seq quantification. Nature biotechnology 34, 525-527 (2016).

61. Salomonis, N., et al. Alternative splicing in the differentiation of human embryonic stem cells into cardiac precursors. PLoS Comput Biol 5, e1000553 (2009).

62. Ashburner, M., et al. Gene ontology: tool for the unification of biology. The Gene Ontology Consortium. Nat Genet 25, 25-29 (2000).

63. The Gene Ontology, C. Expansion of the Gene Ontology knowledgebase and resources. Nucleic Acids Res 45, D331-D338 (2017).

64. Koressaar, T. & Remm, M. Enhancements and modifications of primer design program Primer3. Bioinformatics 23, 1289-1291 (2007).

65. Untergasser, A., et al. Primer3—new capabilities and interfaces. Nucleic Acids Res 40, e115 (2012).

66. Real-time PCR handbook, (Thermo Fisher Scientific Inc., 2016).

67. Schmidt, E. K., Clavarino, G., Ceppi, M. & Pierre, P. SUnSET, a nonradioactive method to monitor protein synthesis. Nat Methods 6, 275-277 (2009).

68. Goodman, C. A., et al. Novel insights into the regulation of skeletal muscle protein synthesis as revealed by a new nonradioactive in vivo technique. FASEB J 25, 1028-1039 (2011).

69. Penna, F., et al. Effect of the specific proteasome inhibitor bortezomib on cancer-related muscle wasting. J Cachexia Sarcopenia Muscle 7, 345-354 (2016).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of treating a muscle contracture in an individual in need thereof, comprising administration of a proteasome inhibitor to said individual.

2. The method of claim 1, wherein said administration results in an improvement in longitudinal muscle growth.

3. The method of claim 1, wherein said muscle contracture is associated with a neuromuscular disorder selected from neonatal brachial plexus injury (NBPI) and cerebral palsy (CP).

4. The method of claim 1, wherein said individual is diagnosed with cerebral palsy and wherein said muscle contracture is characterized by a lower neurologic lesion.

5. The method of claim 1, wherein said individual is diagnosed with neonatal brachial plexus injury, and wherein said muscle contracture is characterized by an upper neurologic lesion.

6. The method of claim 1, wherein said administration results in a decrease in contracture severity in said individual.

7. The method of claim 1, wherein said administration results in increased range of motion in a joint of said individual as compared to prior to administration step.

8. The method of claim 1, wherein said proteasome inhibitor is a 20S proteasome inhibitor, a 26S proteasome inhibitor, or a combination thereof.

9. The method of claim 1, wherein said proteasome inhibitor is a peptide boronate.

10. The method of claim 1, wherein said proteasome inhibitor is selected from Bortezomib, carfilzomib, and combinations thereof.

11. The method of claim 1, wherein said proteasome inhibitor is selected from a peptide aldehyde, a peptide vinyl sulfone, a peptide epoxyketone, a beta lactone inhibitor, and combinations thereof.

12. The method of claim 1, wherein said proteasome inhibitor is a compound that creates a dithiocarbamate complex with metal.

13. The method of claim 1, wherein said proteasome inhibitor is bortezomib, and wherein said proteasome inhibitor is co-administered with a neuroprotective agent.

14. The method of claim 1, wherein said administration occurs during a period of neonatal muscle growth of said individual.

15. The method of claim 1, wherein said administration step occurs at an age selected from less than 10 weeks of age, less than 9 weeks of age, less than 8 weeks of age, less than 7 weeks of age, less than 6 weeks of age, less than 5 weeks of age, less than 4 weeks of age, less than 3 weeks of age, less than 2 weeks of age, or less than 1 week of age.

16. The method of claim 1, wherein said administration is carried out at an interval selected from three times a day, twice a day, once a day, once every other day, once every two days, once every three days, once every four days, once every five days, once every six days, once a week, and once every two weeks.

17. The method of claim 1, wherein said administration step is carried out prior to contracture development, wherein said individual exhibits one or more signs selected from paralysis or weakness of muscles during the neonatal period.

18. A method of improving longitudinal muscle length in an individual in need thereof, comprising administering to said individual a therapeutic dose of a proteasome inhibitor, wherein said administration is limited to a period of time selected from less than 12 weeks, or less than 11 weeks, or less than 10 weeks, or less than nine weeks, or less than eight weeks, or less than seven weeks, or less than six weeks, or less than five weeks, or less than four weeks, or less than three weeks, or less than two weeks, or less than one week.

19. The method of claim 1, wherein said proteasome inhibitor is bortezomib, and wherein said proteasome inhibitor is co-administered with [Gly 14]-Humanin.


SEQUENCE LISTING (8 sequences)

SEQ ID NO: 1 - DNA, 19 bp, Artificial Sequence
  Forward Primer Mymk
atcgctaccaagaggcgtt

SEQ ID NO: 2 - DNA, 20 bp, Artificial Sequence
  Reverse primer Mymk
cacagcacagacaaaccagg

SEQ ID NO: 3 - DNA, 20 bp, Artificial Sequence
  Forward primer GAPDH
tgcgacttcaacagcaactc

SEQ ID NO: 4 - DNA, 20 bp, Artificial Sequence
  Reverse primer GAPDH
gcctctcttgctcagtgtcc

SEQ ID NO: 5 - DNA, 20 bp, Artificial Sequence
  Forward primer Trim63-202
ggagaacctggagaagcagc

SEQ ID NO: 6 - DNA, 20 bp, Artificial Sequence
  Reverse primer Trim63-202
tagggattcgcagcctggaa

SEQ ID NO: 7 - DNA, 22 bp, Artificial Sequence
  Forward primer Atp5j-201
tcagtgcaagtacagagactca

SEQ ID NO: 8 - DNA, 22 bp, Artificial Sequence
  Reverse primer Atp5j-201
gcctgtcgctttgatttgtact